(19) United States
(12) Patent Application Publication
(10) Pub. No.: US 2023/0400767 A1
(43) Pub. Date: Dec. 14, 2023

(54) RADIATION-SENSITIVE RESIN COMPOSITION, METHOD OF FORMING RESIST PATTERN, POLYMER, AND COMPOUND

(71) Applicant: JSR CORPORATION, Tokyo (JP)

(72) Inventors: Takuya OMIYA, Tokyo (JP); Katsuaki NISHIKORI, Tokyo (JP); Kazuya KIRIYAMA, Tokyo (JP); Natsuko KINOSHITA, Tokyo (JP); Tetsurou KANEKO, Tokyo (JP)

(73) Assignee: JSR CORPORATION, Tokyo (JP)

(21) Appl. No.: 18/239,399

(22) Filed: Aug. 29, 2023

Related U.S. Application Data
(63) Continuation of application No. PCT/JP21/48363, filed on Dec. 24, 2021.

(30) Foreign Application Priority Data
Mar. 18, 2021 (JP) 2021-045282

(51) Int. Cl. G03F 7/039; G03F 7/038; G03F 7/004; C08F 212/14; C07C 69/54; C08F 220/18; C08F 220/24; C08F 220/22; C08F 220/30; C08F 220/34

(52) U.S. Cl. CPC G03F 7/039; G03F 7/038; G03F 7/0045; C08F 212/24; C07C 69/54; C08F 220/1805; C08F 220/1806; C08F 220/1807; C08F 220/1808; C08F 220/1809; C08F 220/24; C08F 220/1811; C08F 220/22; C08F 220/301; C08F 220/1812; C08F 220/1818; C08F 220/34

(57) ABSTRACT

A radiation-sensitive resin composition includes: a polymer including a first structural unit represented by formula (1); and a radiation-sensitive acid generator. In the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

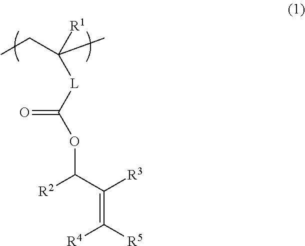

### CROSS-REFERENCE TO RELATED APPLICATIONS

The present application is a continuation application of International Application No. PCT/JP2021/048363, filed Dec. 24, 2021, which claims priority to Japanese Patent Application No. 2021-045282 filed Mar. 18, 2021. The contents of these applications are incorporated herein by reference in their entirety.

### BACKGROUND OF THE INVENTION

### Field of the Invention

The present invention relates to a radiation-sensitive resin composition, a method of forming a resist pattern, a polymer, and a compound.

### Discussion of the Background

A radiation-sensitive resin composition for use in microfabrication by lithography generates an acid at light-exposed regions upon an irradiation with a radioactive ray, e.g.: an electromagnetic wave such as a far ultraviolet ray such as an ArF excimer laser beam (wavelength of 193 nm) or a KrF excimer laser beam (wavelength of 248 nm), or an extreme ultraviolet ray (EUV) (wavelength of 13.5 nm); or a charged particle ray such as an electron beam. A chemical reaction in which the acid serves as a catalyst causes a difference between the light-exposed regions and light-unexposed regions in rates of dissolution in a developer solution, whereby a resist pattern is formed on a substrate.

Such radiation-sensitive resin compositions are required not only to have favorable sensitivity to exposure light such as the extreme ultraviolet ray and the electron beam, but also to result in superiority in LWR (Line Width Roughness) performance, CDU (Critical Dimension Uniformity) performance, and the like.

To meet these requirements, types, molecular structures, and the like of polymers, acid generating agents, and other components which may be used in the radiation-sensitive resin compositions have been investigated, and combinations thereof have been further investigated in detail (see Japanese Unexamined Patent Applications, Publication Nos. 2010-134279, 2014-224984, and 2016-047815).

### SUMMARY OF THE INVENTION

According to an aspect of the present invention, a radiation-sensitive resin composition includes: a polymer including a first structural unit represented by formula (1); and a radiation-sensitive acid generator.

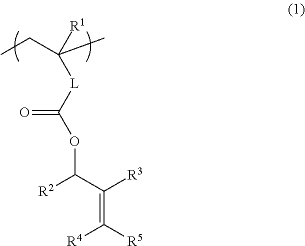

In the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

According to another aspect of the present invention, a method of forming a resist pattern includes: forming a resist film directly or indirectly on a substrate by applying a radiation-sensitive resin composition; exposing the resist film; and developing the resist film exposed. The radiation-sensitive resin composition includes: a polymer including a first structural unit represented by formula (1); and a radiation-sensitive acid generator.

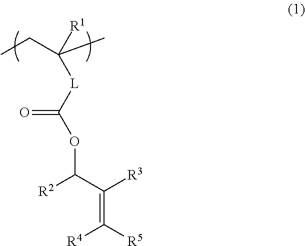

In the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

According to a further aspect of the present invention, a polymer includes a first structural unit represented by formula (1).

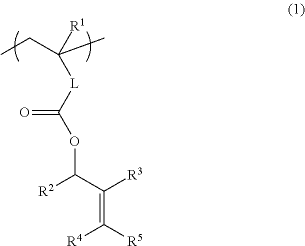

In the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

According to a further aspect of the present invention, a compound is represented by formula (1′).

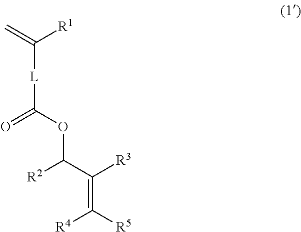

In the formula (1′), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

### DESCRIPTION OF EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Along with further miniaturization of resist patterns, slight changes in exposure and development conditions have come to exert an increasingly larger effect on configurations and generation of defects of resist patterns. Thus, a radiation-sensitive resin composition with a broad process window (a high process latitude) which enables absorption of such slight fluctuations in process conditions is also required.

According to one embodiment of the invention, a radiation-sensitive resin composition contains: a polymer having a first structural unit represented by the following formula (1); and a radiation-sensitive acid generator,

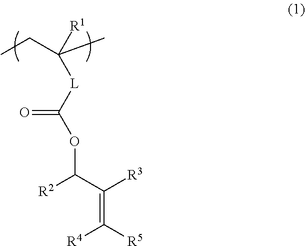

wherein, in the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

According to an other embodiment of the invention, a method of forming a resist pattern includes: applying a radiation-sensitive resin composition directly or indirectly on a substrate; exposing a resist film formed by the applying; and developing the resist film exposed, wherein the radiation-sensitive resin composition contains: a polymer having a first structural unit represented by the following formula (1); and a radiation-sensitive acid generator,

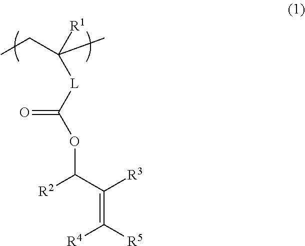

wherein, in the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

Still another embodiment of the invention is a polymer having a first structural unit represented by the following formula (1):

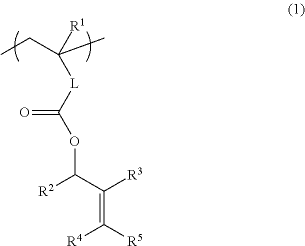

wherein, in the formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

Yet another embodiment of the invention is a compound represented by the following formula (1′):

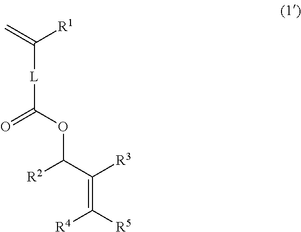

wherein, in the formula (1′), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

The radiation-sensitive resin composition and the method of forming a resist pattern of the embodiments of the present invention enable formation of a resist pattern with favorable sensitivity to exposure light, superiority in LWR performance and CDU performance, and a broad process window. The polymer of the still another embodiment of the present invention can be suitably used as a component of the radiation-sensitive resin composition of the one embodiment of the present invention. The compound of the yet another embodiment of the present invention can be suitably used as a monomer for synthesizing the polymer of the still another embodiment of the present invention. Therefore, these can be suitably used in manufacturing processes of semiconductor devices and the like, in which further progress of miniaturization is expected in the future.

Hereinafter, the radiation-sensitive resin composition, the method of forming a resist pattern, the polymer, and the compound of embodiments of the present invention will be described in detail.

### Radiation-Sensitive Resin Composition

The radiation-sensitive resin composition according to one embodiment of the present invention contains: a polymer (hereinafter, may be also referred to as “(A) polymer” or “polymer (A)”) having a first structural unit represented by the following formula (1), described later; and a radiation-sensitive acid generator (hereinafter, may be also referred to as “(B) acid generator” or “acid generator (B)”). The radiation-sensitive resin composition typically contains an organic solvent (hereinafter, may be also referred to as “(D) organic solvent” or “organic solvent (D)”). The radiation-sensitive resin composition may contain, as a favorable component, an acid diffusion control agent (hereinafter, may be also referred to as “(C) acid diffusion control agent” or “acid diffusion control agent (C)”). The radiation-sensitive resin composition may also contain, within a range not leading to impairment of the effects of the present invention, other optional component(s).

Due to the polymer (A) and the acid generator (B) being contained, the radiation-sensitive resin composition enables a resist pattern to be formed with favorable sensitivity to exposure light, superiority in LWR performance and CDU performance, and a broad process window. Although not necessarily clarified and without wishing to be bound by any theory, the reason for achieving the aforementioned effects by the radiation-sensitive resin composition due to involving such a constitution may be presumed, for example, as in the following. It is considered that due to the first structural unit in the polymer (A) having a certain acid-labile group, described later, acid dissociation efficiency is improved, and consequently, a resist pattern can be formed with favorable sensitivity to exposure light, superiority in LWR performance and CDU performance, and a broad process window.

The radiation-sensitive resin composition can be prepared, for example, by mixing the polymer (A) and the acid generator (B), as well as the acid diffusion control agent (C), the organic solvent (D) and/or the other optional component(s), which is/are added as needed, in a certain ratio, and preferably filtering a thus resulting mixture through a membrane filter having a pore size of no greater than 0.2 m.

Each component contained in the radiation-sensitive resin composition is described below.

### (A) Polymer

The polymer (A) has a first structural unit (hereinafter, may be also referred to as “structural unit (I)”) represented by the following formula (1), described later. The radiation-sensitive resin composition may contain one, or two or more types of the polymer (A).

The polymer (A) preferably further has a second structural unit (hereinafter, may be also referred to as “structural unit (II)”) which includes a phenolic hydroxy group. The polymer (A) preferably further has a third structural unit (hereinafter, may be also referred to as “structural unit (III)”) which is a structural unit other than the structural unit (I) and includes an acid-labile group. The polymer (A) may further have other structural unit(s) (hereinafter, may be also referred to as simply “other structural unit(s)”) aside from the structural units (I) to (III).

The lower limit of a proportion of the polymer (A) in the radiation-sensitive resin composition with respect to total components other than the organic solvent (D) contained in the radiation-sensitive resin composition is preferably 50% by mass, more preferably 70% by mass, and still more preferably 80% by mass. The upper limit of the proportion is preferably 99% by mass, and more preferably 95% by mass.

The lower limit of a polystyrene-equivalent weight average molecular weight (Mw) of the polymer (A) as determined by gel permeation chromatography (GPC) is preferably 1,000, more preferably 3,000, still more preferably 5,000, yet more preferably 6,000, and particularly preferably 7,000. The upper limit of the Mw is preferably 50,000, more preferably 30,000, still more preferably 20,000, yet more preferably 15,000, and particularly preferably 10,000. When the Mw of the polymer (A) falls within the above range, coating characteristics of the radiation-sensitive resin composition may be improved. The Mw of the polymer (A) can be adjusted by, for example, regulating the type, the amount, and the like of a polymerization initiator used in synthesis of the polymer (A).

The upper limit of a ratio (hereinafter may be also referred to as “dispersity index” or “Mw/Mn”) of the Mw to a polystyrene-equivalent number average molecular weight (Mn) of the polymer (A) as determined by GPC is preferably 2.50, more preferably 2.00, and still more preferably 1.75. The lower limit of the ratio is typically 1.00, preferably 1.10, more preferably 1.20, and still more preferably 1.30.

Method for Measuring Mw and Mn

As referred to herein, the Mw and Mn of the polymer (A) are values measured by using gel permeation chromatography (GPC) under the following conditions.

GPC columns: “G2000 HXL”×2, “G3000 HXL”×1, and “G4000 HXL”×1, available from Tosoh Corporation
column temperature: 40° C.
elution solvent: tetrahydrofuran
flow rate: 1.0 mL/min
sample concentration: 1.0% by mass
amount of injected sample: 100 uL
detector: differential refractometer
standard substance: mono-dispersed polystyrene

The polymer (A) can be synthesized by, for example, polymerizing a monomer that gives each structural unit in accordance with a well-known procedure.

Each structural unit contained in the polymer (A) is described below.

Structural Unit (I)

The structural unit (I) is a structural unit represented by the following formula (1). The structural unit (I) is a structural unit that includes an acid-labile group. The “acid-labile” group as referred to means a group that substitutes for a hydrogen atom in a carboxy group, and is capable of being dissociated by an action of an acid to give a carboxy group. In the following formula (1), a group (—CH(R²)—C(R³)═CR⁴R⁵) which bonds to an ethereal oxygen atom in a carbonyloxy group is an acid-labile group (hereinafter, may be also referred to as “acid-labile group (a)”). The acid-labile group (a) is dissociated by an action of the acid generated from the acid generator (B), and/or the like upon exposure, whereby a difference is generated in the solubility of the polymer (A) in the developer solution, between light-exposed regions and light-unexposed regions, and thus forming a resist pattern is enabled. The polymer (A) having the structural unit (I) enables a resist pattern to be formed with favorable sensitivity to exposure light, superiority in LWR performance and CDU performance, and a broad process window. The polymer (A) may have one, or two or more types of the structural unit (I).

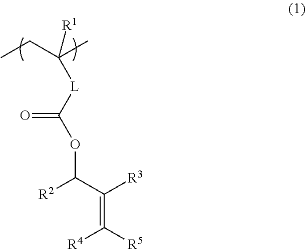

In the above formula (1), R¹ represents a hydrogen atom, a fluorine atom, a methyl group, or a trifluoromethyl group; R², R³, and R⁴ each independently represent a hydrogen atom or a monovalent organic group having 1 to 20 carbon atoms; R⁵ represents a monovalent organic group having 1 to 20 carbon atoms; and L represents a single bond or a divalent organic group having 1 to 20 carbon atoms.

The number of “carbon atoms” as referred to herein means the number of carbon atoms constituting a group. The “organic group” as referred to herein means a group that includes at least one carbon atom.

The monovalent organic group having 1 to 20 carbon atoms which may be represented by R², R³, R⁴, or R⁵ is exemplified by: a monovalent hydrocarbon group having 1 to 20 carbon atoms; a group (hereinafter, may be also referred to as “group (α)”) that contains a divalent heteroatom-containing group between two adjacent carbon atoms of this hydrocarbon group; a group (hereinafter, may be also referred to as “group (β)”) obtained by substituting a part or all of hydrogen atoms included in the hydrocarbon group or the group (α) with a monovalent heteroatom-containing group; a group (hereinafter, may be also referred to as “group (γ)”) obtained by combining the hydrocarbon group, the group (α), or the group (β) with a divalent heteroatom-containing group; and the like.

The “hydrocarbon group” as referred to herein may include a chain hydrocarbon group, an alicyclic hydrocarbon group, and an aromatic hydrocarbon group. The “hydrocarbon group” may be either a saturated hydrocarbon group or an unsaturated hydrocarbon group. The “chain hydrocarbon group” as referred to herein means a hydrocarbon group not containing a cyclic structure but being constituted with only a chain structure, and both a linear hydrocarbon group and a branched chain hydrocarbon group may be included. The “alicyclic hydrocarbon group” as referred to herein means a hydrocarbon group that contains, as a ring structure, not an aromatic ring structure but an alicyclic structure alone, and may include both a monocyclic alicyclic hydrocarbon group and a polycyclic alicyclic hydrocarbon group. However, it is not necessary for the alicyclic hydrocarbon group to be constituted with only an alicyclic structure; it may contain a chain structure in a part thereof. The “aromatic hydrocarbon group” as referred to herein means a hydrocarbon group that contains an aromatic ring structure as a ring structure. However, it is not necessary for the aromatic hydrocarbon group to be constituted with only an aromatic ring structure; it may contain a chain structure or an alicyclic structure in a part thereof.

The monovalent hydrocarbon group having 1 to 20 carbon atoms is exemplified by a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent chain hydrocarbon group having 1 to 20 carbon atoms include: alkyl groups such as a methyl group, an ethyl group, an n-propyl group, an i-propyl group, an n-butyl group, a sec-butyl group, an isobutyl group, and a tert-butyl group; alkenyl groups such as an ethenyl group, a propenyl group, a butenyl group, and a 2-methylpropa-1-en-1-yl group; alkynyl groups such as an ethynyl group, a propynyl group, and a butynyl group; and the like.

Examples of the monovalent alicyclic hydrocarbon group having 3 to 20 carbon atoms include: monocyclic alicyclic saturated hydrocarbon groups such as a cyclopentyl group and a cyclohexyl group; polycyclic alicyclic saturated hydrocarbon groups such as a norbornyl group, an adamantyl group, a tricyclodecyl group, and a tetracyclododecyl group; monocyclic alicyclic unsaturated hydrocarbon groups such as a cyclopentenyl group and a cyclohexenyl group; polycyclic alicyclic unsaturated hydrocarbon groups such as a norbornenyl group, a tricyclodecenyl group, and a tetracyclododecenyl group; and the like.

Examples of the monovalent aromatic hydrocarbon group having 6 to 20 carbon atoms include: aryl groups such as a phenyl group, a tolyl group, a xylyl group, a naphthyl group, and an anthryl group; aralkyl groups such as a benzyl group, a phenethyl group, a naphthylmethyl group, and an anthrylmethyl group; and the like.

The heteroatom that may constitute the monovalent or divalent heteroatom-containing group is exemplified by an oxygen atom, a nitrogen atom, a sulfur atom, a phosphorus atom, a silicon atom, a halogen atom, and the like. Examples of the halogen atom include a fluorine atom, a chlorine atom, a bromine atom, and an iodine atom.

Examples of the monovalent heteroatom-containing group include the halogen atom, a hydroxy group, an alkoxy group, a carboxy group, a cyano group, an amino group (—NR₂), a sulfanyl group, and the like, wherein Rs may be the same or different, and each represents a hydrogen atom or the monovalent hydrocarbon group. Of these, the halogen atom, a hydroxy group, an alkoxy group, or an amino group is preferred, and a fluorine atom, a hydroxy group, a methoxy group, or a dimethylamino group is more preferred.

Examples of the divalent heteroatom-containing group include —O—, —CO—, —S—, —CS—, —SO₂—, —NR′—, groups in which at least two of the aforementioned groups are combined (for example, —O—C—O—, etc.), and the like. R′ represents a hydrogen atom or a monovalent hydrocarbon group.

Examples of the divalent organic group having 1 to 20 carbon atoms which may be represented by L include groups obtained by removing one hydrogen atom from the groups exemplified as the monovalent organic group having 1 to 20 carbon atoms which may be represented as R², R³, R⁴, or R⁵, and the like.

In light of copolymerizability with a monomer that gives the structural unit (I), R¹ represents preferably a hydrogen atom or a methyl group, and more preferably a methyl group.

In regard to R², the case of R² representing the monovalent organic group having 1 to 20 carbon atoms enables improving the sensitivity to exposure light, the LWR performance, the CDU performance, and the process window more than the case of R² representing a hydrogen atom. In the case of R² representing the monovalent organic group having 1 to 20 carbon atoms, the acid-labile group (a) bonds to the ethereal oxygen atom in the carbonyloxy group by a secondary carbon atom. On the other hand, in the case of R² representing a hydrogen atom, the acid-labile group (a) bonds to the ethereal oxygen atom in the carbonyloxy group by a primary carbon atom. While a limited interpretation is not desired, it is presumed that due to the difference in level of the carbon atom in the acid-labile group (a) which bonds to the ethereal oxygen atom in the carbonyloxy group, the above-described further beneficial effects are achieved.

In other words, in light of further improving the sensitivity to exposure light and the LWR performance, CDU performance, and process window, R² represents preferably the monovalent organic group having 1 to 20 carbon atoms, and more preferably the monovalent hydrocarbon group having 1 to 20 carbon atoms or the group (group (β)) obtained by substituting a part or all of hydrogen atoms contained in this hydrocarbon group with the monovalent heteroatom-containing group.

In the case in which R² represents the monovalent hydrocarbon group having 1 to 20 carbon atoms, this hydrocarbon group is preferably the chain hydrocarbon group, the alicyclic hydrocarbon group, or the aromatic hydrocarbon group, more preferably the alkyl group, the alkenyl group, the monocyclic alicyclic saturated hydrocarbon group, or the aryl group, and still more preferably a methyl group, an ethyl group, an isopropyl group, a tert-butyl group, a 2-methylpropa-1-en-1-yl group, a cyclohexyl group, or a phenyl group.

In the case in which R² represents the group (β), the group (β) is preferably a group obtained by substituting a part or all of hydrogen atoms contained in the hydrocarbon group with the halogen atom or an alkoxy group, more preferably a group obtained by substituting a part or all of hydrogen atoms contained in the hydrocarbon group with a fluorine atom or a methoxy group, and still more preferably a trifluoromethyl group, a 4,4-difluorocyclomethyl group, a 4-fluorophenyl group, a 4-methoxyphenyl group, or a 2,2,2-trifluoro-1,1-dimethylethyl group.

R³ represents preferably a hydrogen atom or the monovalent hydrocarbon group having 1 to 20 carbon atoms, more preferably a hydrogen atom or the monovalent chain hydrocarbon group having 1 to 20 carbon atoms, still more preferably a hydrogen atom or the alkyl group, and yet more preferably a hydrogen atom or a methyl group.

R⁴ represents preferably a hydrogen atom or the monovalent hydrocarbon group having 1 to 20 carbon atoms, more preferably a hydrogen atom or the monovalent chain hydrocarbon group having 1 to 20 carbon atoms, still more preferably a hydrogen atom or the alkyl group, and yet more preferably a hydrogen atom or a methyl group.

R⁵ represents preferably the monovalent hydrocarbon group having 1 to 20 carbon atoms or the group (group (β)) obtained by substituting a part or all of hydrogen atoms contained in this hydrocarbon group with the monovalent heteroatom-containing group.

In the case in which R⁵ represents the monovalent hydrocarbon group having 1 to 20 carbon atoms, this hydrocarbon group is preferably a chain hydrocarbon group or an aromatic hydrocarbon group, more preferably an alkenyl group or an aryl group, and still more preferably a 3-methylbuta-2-en-1-yl group, a phenyl group, or a 9-anthryl group.

In the case in which R⁵ represents the group (β), this group (β) is preferably a group obtained by substituting a part or all of hydrogen atoms contained in the hydrocarbon group with the halogen atom, a hydroxy group, an alkoxy group, or an amino group, more preferably a group obtained by substituting a part or all of hydrogen atoms contained in the aromatic hydrocarbon group with a fluorine atom, a hydroxy group, a methoxy group, or a dimethylamino group, and still more preferably a 4-fluorophenyl group, a 4-hydroxyphenyl group, a 4-methoxyphenyl group, or a 4-dimethylaminophenyl group.

L represents preferably a single bond.

As the structural unit (I), structural units (hereinafter, may be also referred to as “structural units (I-1) to (I-30)”) represented by the following formulae (1-1) to (1-30) are preferred, and the structural units (I-2) to (I-30) are further preferred.

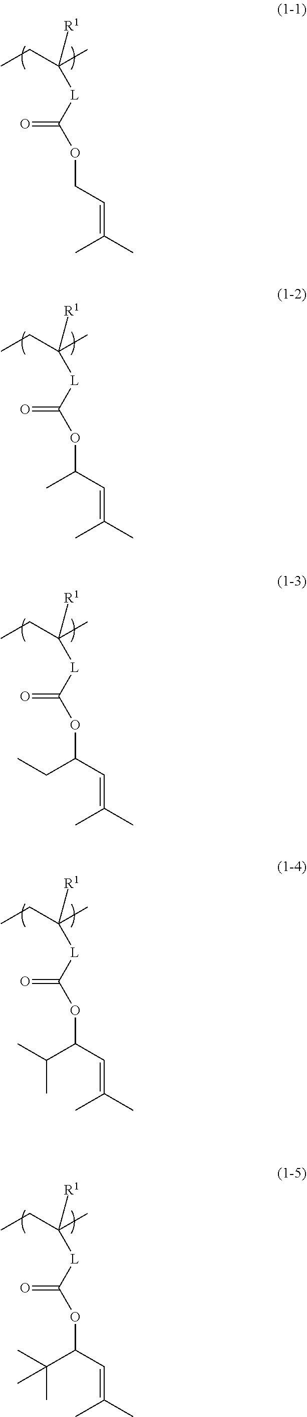

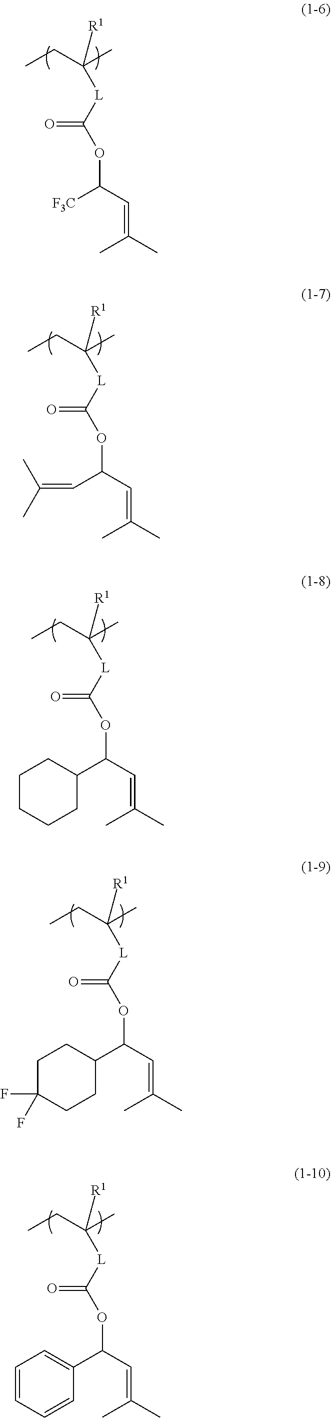

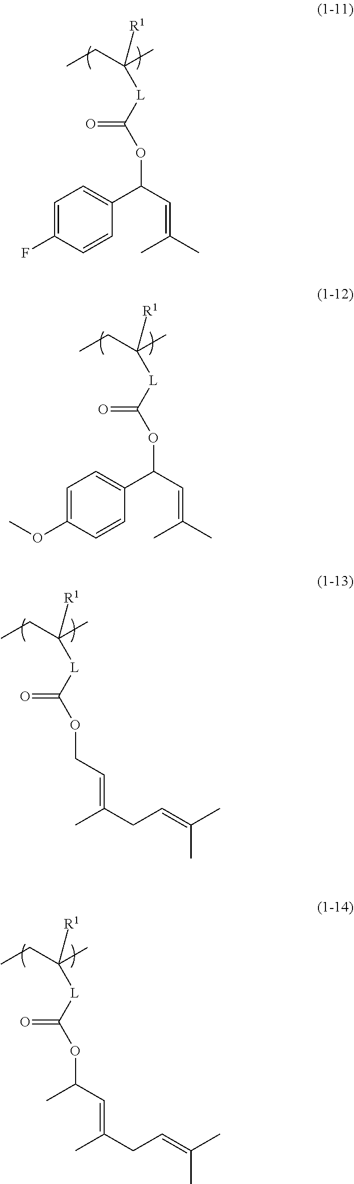

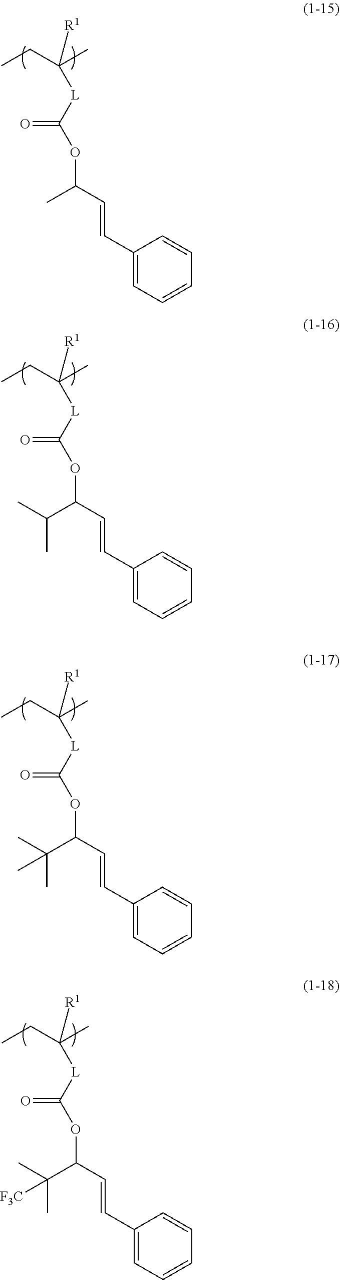

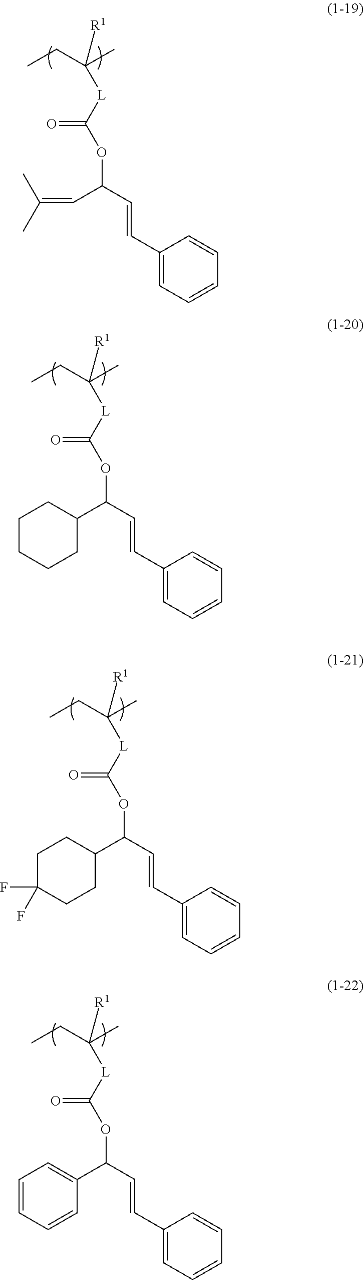

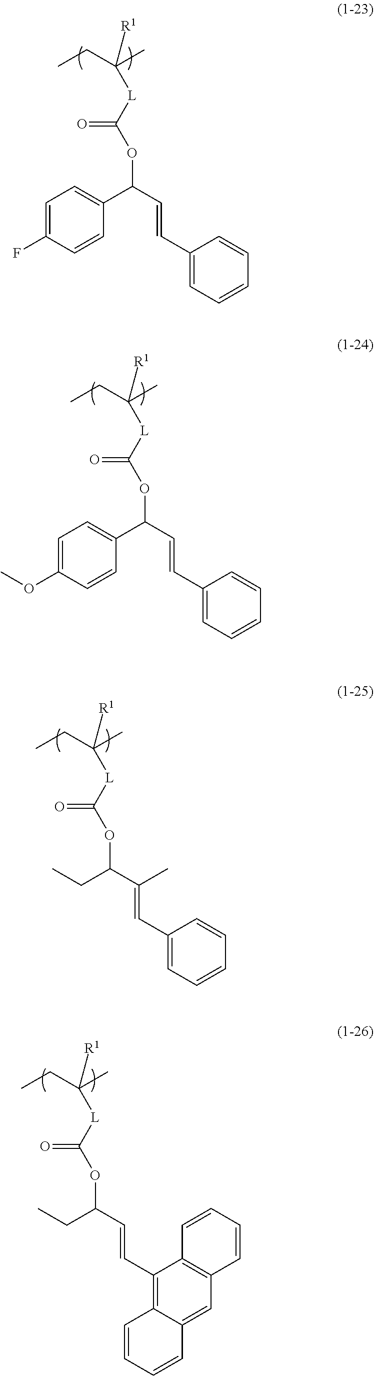

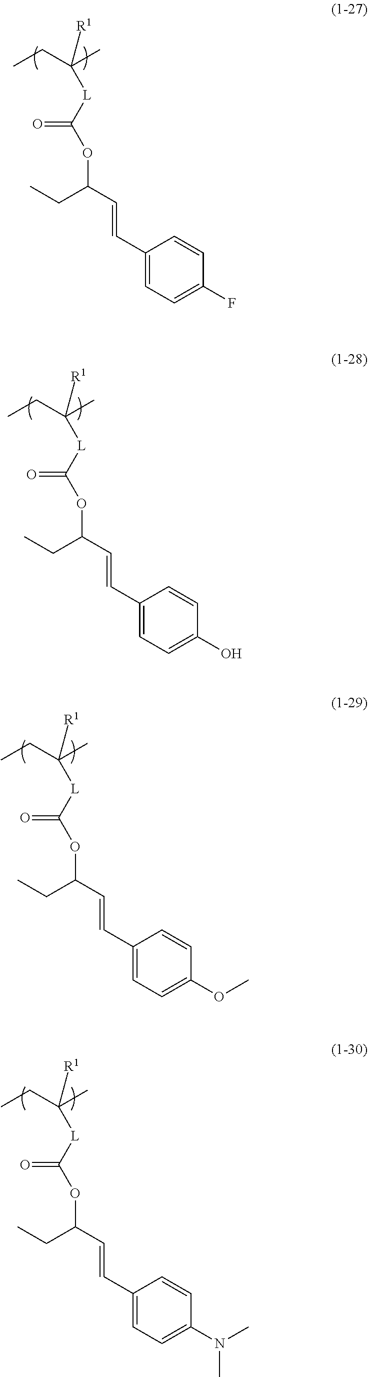

The lower limit of a proportion of the structural unit (I) in the polymer (A) with respect to total structural units constituting the polymer (A) is preferably 5 mol %, more preferably 10 mol %, still more preferably 15 mol %, and yet more preferably 20 mol %. The upper limit of the proportion is preferably 80 mol %, more preferably 75 mol %, still more preferably 70 mol %, and yet more preferably 65 mol %. When the proportion of the structural unit (I) falls within the above range, the sensitivity to exposure light of and the LWR performance, CDU performance, and process window resulting from the radiation-sensitive resin composition can be further improved.

Structural Unit (II)

The structural unit (II) is a structural unit including a phenolic hydroxy group. The “phenolic hydroxy group” as referred to is not limited to a hydroxy group directly bonding to a benzene ring, and means any hydroxy group directly bonding to an aromatic ring in general. The polymer (A) may have one, or two or more types of the structural unit (II).

In a case of conducting a KrF exposure, an EUV exposure, or an electron beam exposure, the sensitivity of the radiation-sensitive resin composition to exposure light can be further enhanced due to the polymer (A) having the structural unit (II). Therefore, in the case in which the polymer (A) has the structural unit (II), the radiation-sensitive resin composition can be suitably used as a radiation-sensitive resin composition for the KrF exposure, the EUV exposure, or the electron beam exposure.

Examples of the structural unit (II) include structural units (hereinafter, may be also referred to as “structural units (II-1) to (II-19)”) represented by the following formulae (2-1) to (2-19), and the like.

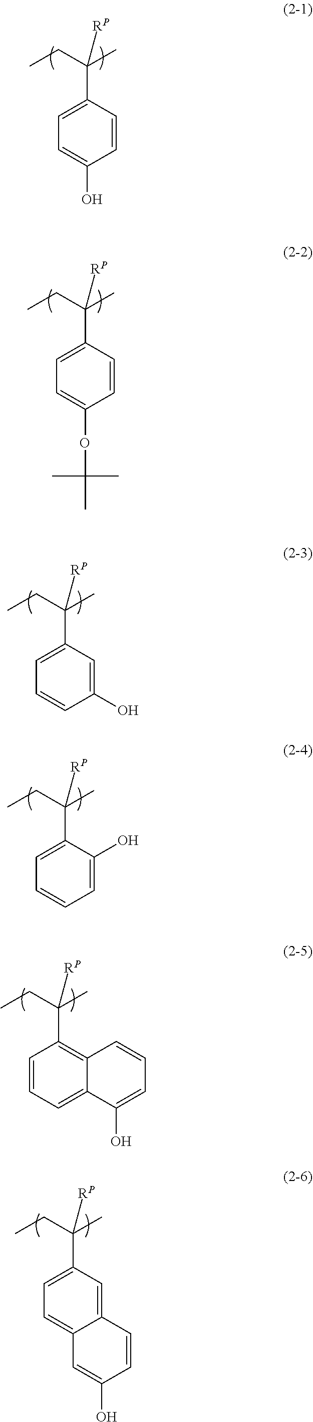

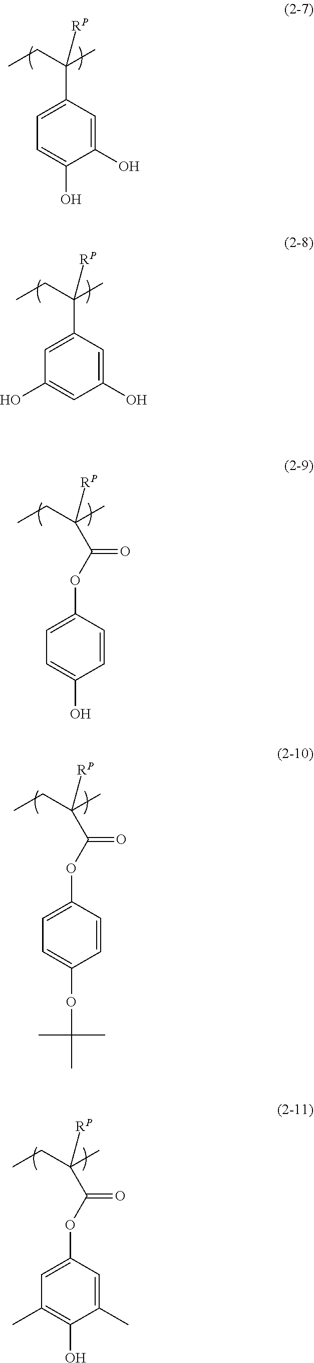

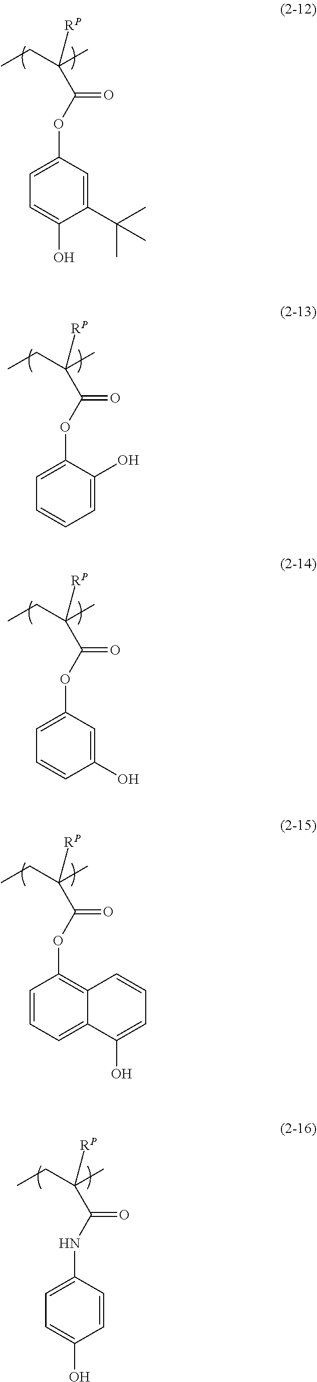

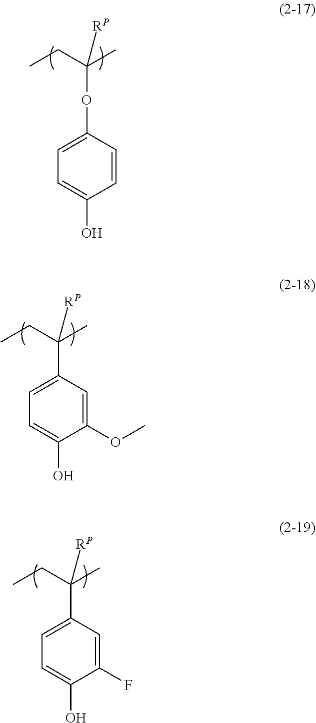

In each of the above formulae (2-1) to (2-19), R^{P} represents a hydrogen atom, a fluorine atom, a methyl group, or a trifluoromethyl group.

In light of copolymerizability of a monomer that gives the structural unit (II), R^{P} represents preferably a hydrogen atom or a methyl group.

As the structural unit (II), one of the structural units (II-1) to (II-3), (II-6) to (II-11), (II-13), and (II-14), or a combination thereof is preferred. In the case of the combination, a combination of two types thereof is preferred, and a combination of the structural unit (II-1) with one type from the structural units (II-2), (II-3), (II-6) to (II-11), (II-13), and (II-14) is further preferred.

In the case in which the polymer (A) has the structural unit (II), the lower limit of a proportion of the structural unit (II) in the polymer (A) with respect to the total structural units constituting the polymer (A) is preferably 20 mol %, more preferably 30 mol %, and still more preferably 40 mol %. The upper limit of the proportion is preferably 80 mol %, more preferably 70 mol %, and still more preferably 65 mol %.

Examples of a monomer that gives the structural unit (II) include a monomer obtained by substituting for a hydrogen atom in a phenolic hydroxy group (—OH) with an acetyl group or the like. In this case, the polymer (A) having the structural unit (II) can be synthesized by, for example, polymerizing the monomer, and then carrying out a hydrolysis reaction on a polymerization reaction product thus obtained, in the presence of a base such as an amine.

Structural Unit (III)

The structural unit (III) is a structural unit other than the structural unit (I) and includes an acid-labile group. The acid-labile group (hereinafter, may be also referred to as “acid-labile group (b)”) included in the structural unit (III) is different from the acid-labile group (a) included in the structural unit (I). The polymer (A) may have one, or two or more types of the structural unit (III).

Examples of the structural unit (III) include structural units (hereinafter, may be also referred to as “structural units (III-1) to (III-3)”) represented by the following formulae (3-1) to (3-3), and the like. It is to be noted that in the following formula (3-1), —C(R^{X})(R^{Y})(R^{Z}) bonding to an ethereal oxygen atom derived from the carboxy group corresponds to the acid-labile group (b).

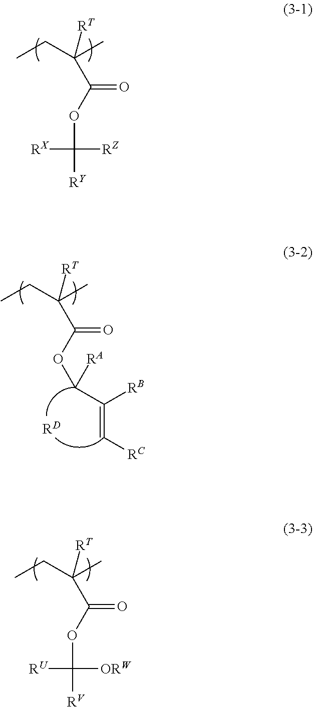

In each of the above formulae (3-1), (3-2), and (3-3), each R^{T} independently represents a hydrogen atom, a fluorine atom, a methyl group, or a trifluoromethyl group.

In the above formula (3-1), R^{X} represents a substituted or unsubstituted monovalent hydrocarbon group having 1 to 20 carbon atoms; and R^{Y} and R^{Z} each independently represent a monovalent hydrocarbon group having 1 to 20 carbon atoms, or R^{Y} and R^{Z} taken together represent a saturated alicyclic structure having 3 to 20 ring atoms together with the carbon atom to which R^{Y} and R^{Z} bond.

In the above formula (3-2), R^{A} represents a hydrogen atom; R^{B} and R^{C} each independently represent a hydrogen atom or a monovalent hydrocarbon group having 1 to 20 carbon atoms; and R^{D} represents a divalent hydrocarbon group having 1 to 20 carbon atoms constituting an unsaturated alicyclic structure having 4 to 20 ring atoms together with the carbon atoms to which R^{A}, R^{B}, and R^{C} each bond.

In the above formula (3-3), R^{U} and R^{V} each independently represent a hydrogen atom or a monovalent hydrocarbon group having 1 to 20 carbon atoms, and R^{W} represents a monovalent hydrocarbon group having 1 to 20 carbon atoms, or R^{U} and R^{V} taken together represent an alicyclic structure having 3 to 20 ring atoms together with the carbon atom to which R^{U} and R^{V} bond, or R^{U} and R^{W} taken together represent an aliphatic heterocyclic structure having 4 to 20 ring atoms together with the carbon atom to which R^{U} bonds and the oxygen atom to which R^{W} bonds.

As referred to herein, the number of “ring atoms” means the number of atoms constituting the ring structure, and in a case of a polycyclic ring, the number of “ring atoms” means the number of atoms constituting the polycyclic ring.

Examples of the monovalent hydrocarbon group having 1 to 20 carbon atoms which may be represented by R^{X}, R^{Y}, R^{Z}, R^{B}, R^{C}, R^{U}, R^{V}, or R^{W} include groups similar to the groups exemplified as the monovalent hydrocarbon group having 1 to 20 carbon atoms among the monovalent organic groups having 1 to 20 carbon atoms which may be represented by R², R³, or R⁴ in the above formula (1), and the like.

Examples of the substituent which may be contained in the hydrocarbon group represented by R^{X} include halogen atoms such as a fluorine atom, a hydroxy group, a carboxy group, a cyano group, a nitro group, an alkoxy group, an alkoxycarbonyl group, an alkoxycarbonyloxy group, an acyl group, an acyloxy group, and the like. Of these, the halogen atom or the alkoxy group is preferred, and a fluorine atom or a methoxy group is more preferred.

Examples of the saturated alicyclic structure having 3 to 20 ring atoms which may be represented by R^{Y} and R^{Z} taken together, together with the carbon atom to which R^{Y} and R^{Z} bond, include: monocyclic saturated alicyclic structures such as a cyclopropane structure, a cyclobutene structure, a cyclopentane structure, and a cyclohexane structure; polycyclic saturated alicyclic structures such as a norbornane structure and an adamantane structure; and the like.

Examples of the divalent hydrocarbon group having 1 to 20 carbon atoms represented by R^{D} include groups obtained by removing one hydrogen atom from the groups exemplified as the monovalent hydrocarbon atom having 1 to 20 carbon atoms, among the monovalent organic groups having 1 to 20 carbon atoms which may be represented by R², R³, or R⁴ in the above formula (1), and the like.

Examples of the unsaturated alicyclic ring structure having 4 to 20 ring atoms represented by R^{D}, together with the carbon atoms to which R^{A}, R^{B}, and R^{C} each bond, include: monocyclic unsaturated alicyclic structures such as a cyclobutene structure, a cyclopentene structure, and a cyclohexene structure; polycyclic unsaturated alicyclic structures such as a norbornene structure; and the like.

Examples of the alicyclic structure having 3 to 20 ring atoms which may be represented by R^{U} and R^{V} taken together, together with the carbon atom to which R^{U} and R^{V} bond include: monocyclic saturated alicyclic structures such as a cyclopropane structure, a cyclobutane structure, a cyclopentane structure, and a cyclohexane structure; polycyclic saturated alicyclic structures such as a norbornane structure, an adamantane structure, a tricyclodecane structure, and a tetracyclododecane structure; monocyclic unsaturated alicyclic structures such as a cyclopropene structure, a cyclobutene structure, a cyclopentene structure, and a cyclohexene structure; polycyclic unsaturated alicyclic structures such as a norbornene structure, a tricyclodecene structure, and a tetracyclododecene structure; and the like.

Examples of the aliphatic heterocyclic structure having 4 to 20 ring atoms which may be represented by R^{U} and R^{W} taken together, together with the carbon atom to which R^{U} bonds and the oxygen atom to which R^{W} bonds include saturated oxygen-containing heterocyclic structures such as an oxacyclobutane structure, an oxacyclopentane structure, and an oxacyclohexane structure; unsaturated oxygen-containing heterocyclic structures such as an oxacyclobutene structure, an oxacyclopentene structure, and an oxacyclohexene structure; and the like.

In light of copolymerizability of a monomer that gives the structural unit (II), R^{T} represents preferably a hydrogen atom or a methyl group.

R^{X} represents preferably a substituted or unsubstituted chain hydrocarbon group or a substituted or unsubstituted aromatic hydrocarbon group, more preferably an unsubstituted chain hydrocarbon group or a substituted or unsubstituted aromatic hydrocarbon group, still more preferably an unsubstituted alkyl group or a substituted or unsubstituted aryl group, and yet more preferably a methyl group, an ethyl group, an i-propyl group, a tert-butyl group, a phenyl group, a 4-methoxyphenyl group, or a 4-trifluoromethylphenyl group.

R^{Y} and R^{Z} each represent preferably the chain hydrocarbon group, more preferably the alkyl group, and still more preferably a methyl group.

Furthermore, it may be also preferred that R^{Y} and R^{Z} taken together represent the saturated alicyclic structure having 3 to 20 ring atoms, together with the carbon atom to which R^{Y} and R^{Z} bond. The saturated alicyclic structure is preferably a cyclopentane structure, a cyclohexane structure, an adamantane structure, or a tetracyclododecane structure.

R^{B} represents preferably a hydrogen atom.

R^{C} represents preferably the chain hydrocarbon group, more preferably the alkyl group, and still more preferably a methyl group.

The unsaturated alicyclic structure having 4 to 20 ring atoms represented by R^{D}, together with the carbon atoms to which R^{A}, R^{B}, and R^{C} each bond is preferably the monocyclic unsaturated alicyclic structure, and more preferably a cyclohexene structure.

The structural unit (III) is preferably the structural unit (III-1) or the structural unit (III-2).

As the structural unit (III-1), structural units (hereinafter, may be also referred to as “structural units (III-1-1) to (III-1-13)”) represented by the following formulae (3-1-1) to (3-1-13) are preferred.

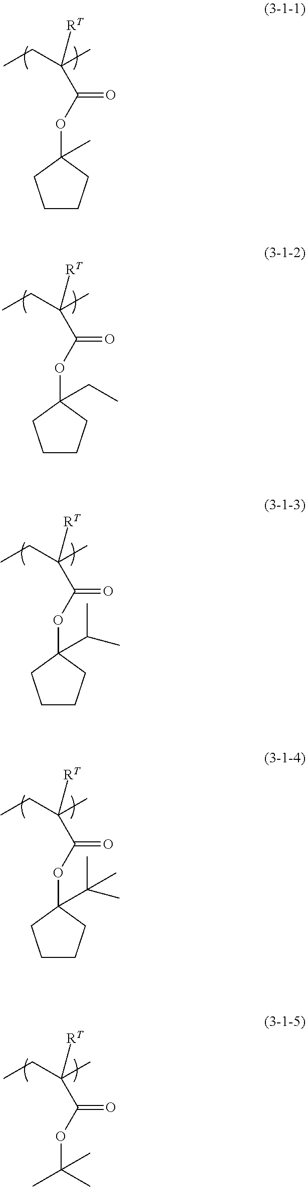

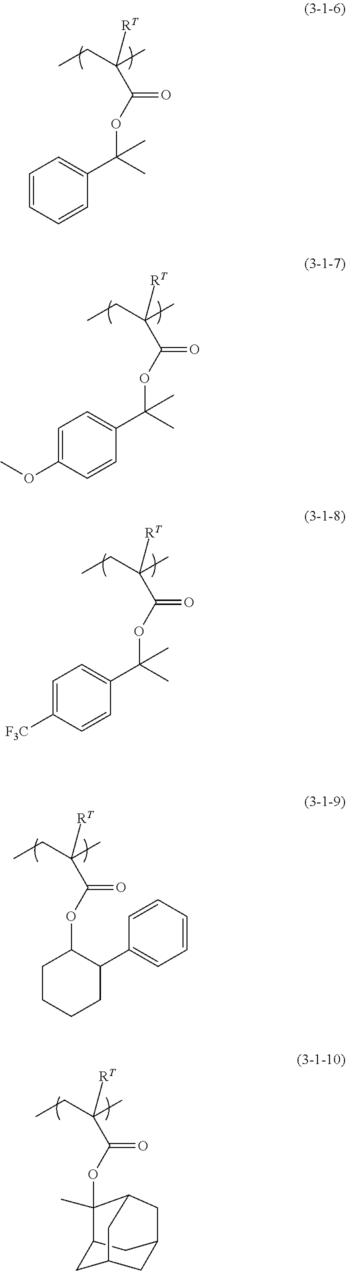

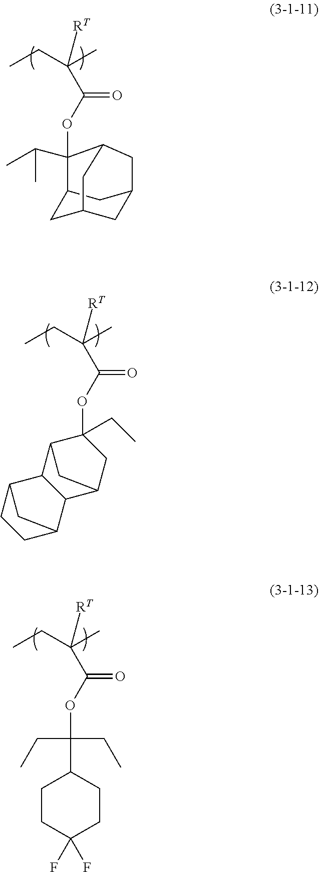

In the above formulae (3-1-1) to (3-1-13), R^{T} is as defined in the above formula (3-1).

The structural unit (III-2) is preferably a structural unit (hereinafter, may be also referred to as “structural unit (III-2-1)”) represented by the following formula (3-2-1).

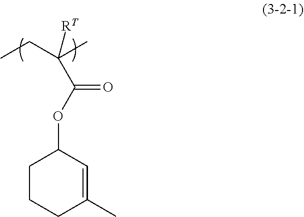

In the above formula (3-2-1), R^{T} is as defined in the above formula (3-2).

In the case in which the polymer (A) has the structural unit (III), the lower limit of a proportion of the structural unit (III) in the polymer (A) with respect to the total structural units constituting the polymer (A) is preferably 5 mol %, more preferably 10 mol %, and still more preferably 15 mol %. The upper limit of the proportion is preferably 50 mol %, more preferably 40 mol %, and still more preferably 30 mol %.

Other Structural Unit(s)

The other structural unit(s) may be exemplified by a structural unit (hereinafter, may be also referred to as “structural unit (IV)”) that includes an alcoholic hydroxy group; a structural unit (hereinafter, may be also referred to as “structural unit (V)”) including a lactone structure, a cyclic carbonate structure, a sultone structure, or a combination thereof; a structural unit (hereinafter, may be also referred to as “structural unit (VI)”) that generates an acid upon an exposure as described in the section “(B) Acid Generator” below; and the like. The polymer (A) may have one, or two or more types of the other structural unit(s).

Structural unit (IV)

The structural unit (IV) is a structural unit that includes an alcoholic hydroxy group.

Examples of the structural unit (IV) include structural units represented by the following formulae, and the like.

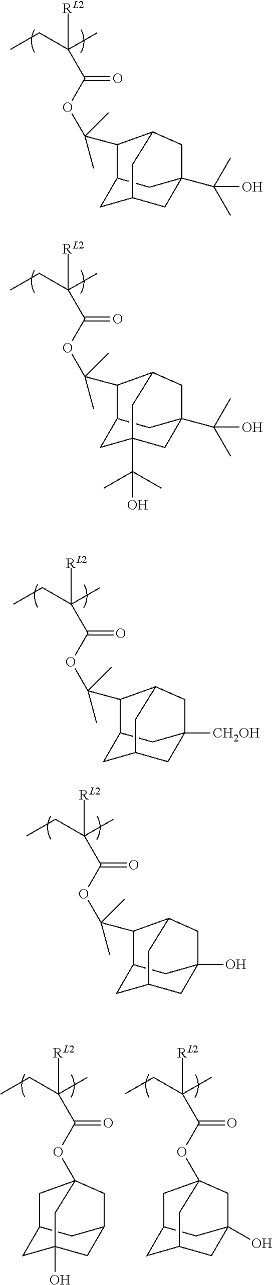

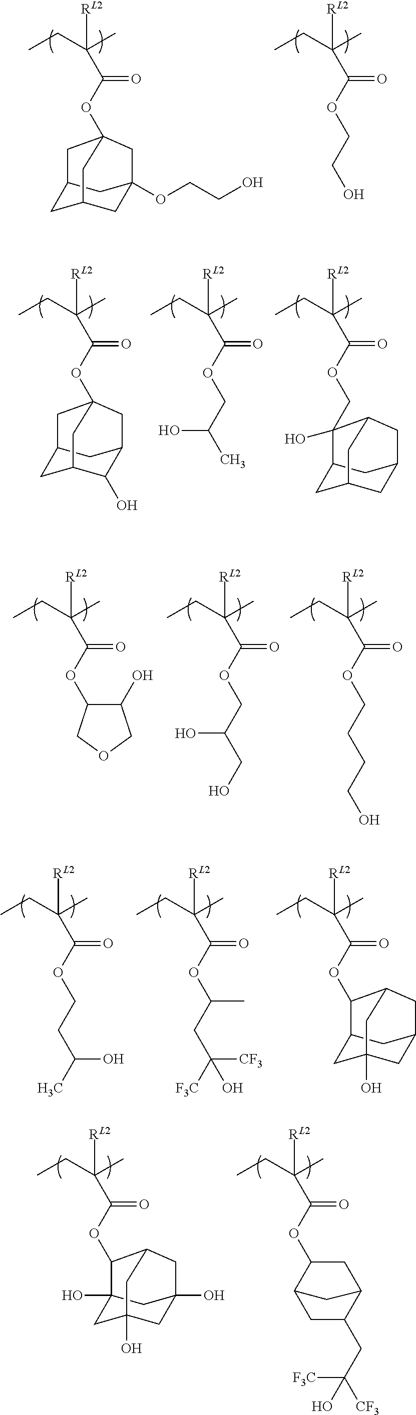

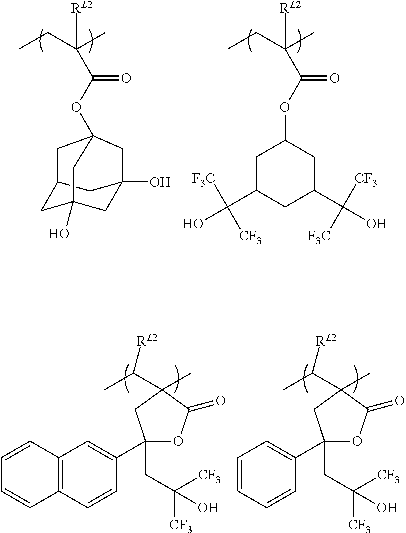

In each of the above formulae, R^{L2} represents a hydrogen atom, a fluorine atom, a methyl group, or a trifluoromethyl group.

In the case in which the polymer (A) has the structural unit (IV), the lower limit of a proportion of the structural unit (IV) contained with respect to the total structural units constituting the polymer (A) is preferably 1 mol %, more preferably 5 mol %, and still more preferably 10 mol %. The upper limit of the proportion is preferably 40 mol %, more preferably 35 mol %, and still more preferably 30 mol %.

Structural unit (V)

The structural unit (V) is a structural unit that includes a lactone structure, a cyclic carbonate structure, a sultone structure, or a combination thereof.

Examples of the structural unit (V) include structural units represented by the following formulae, and the like.

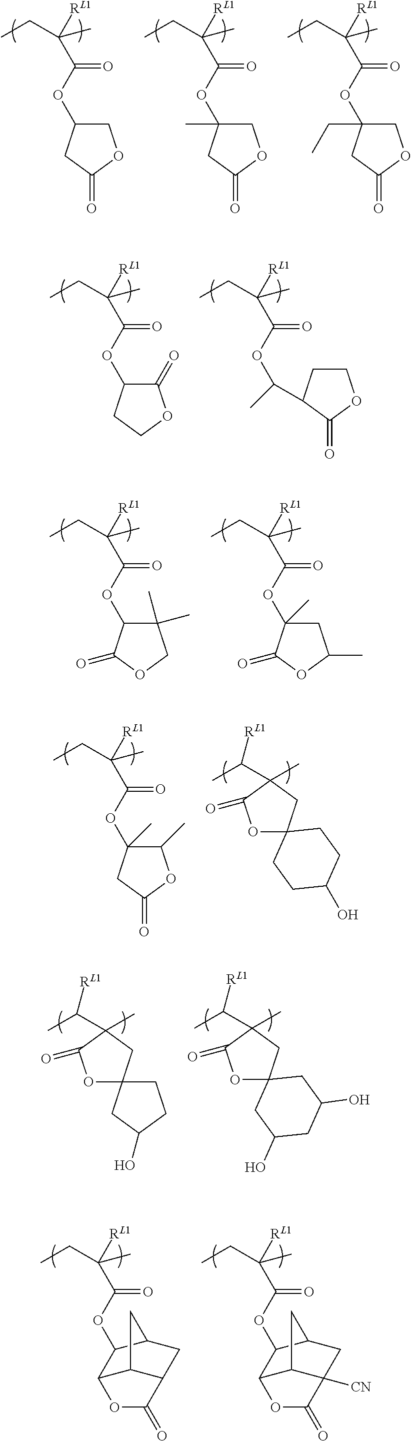

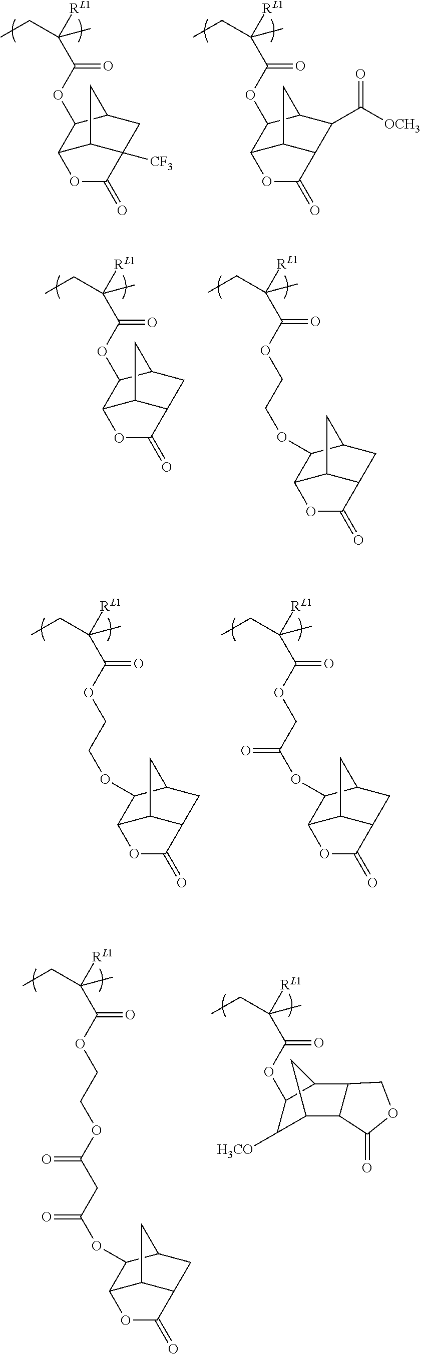

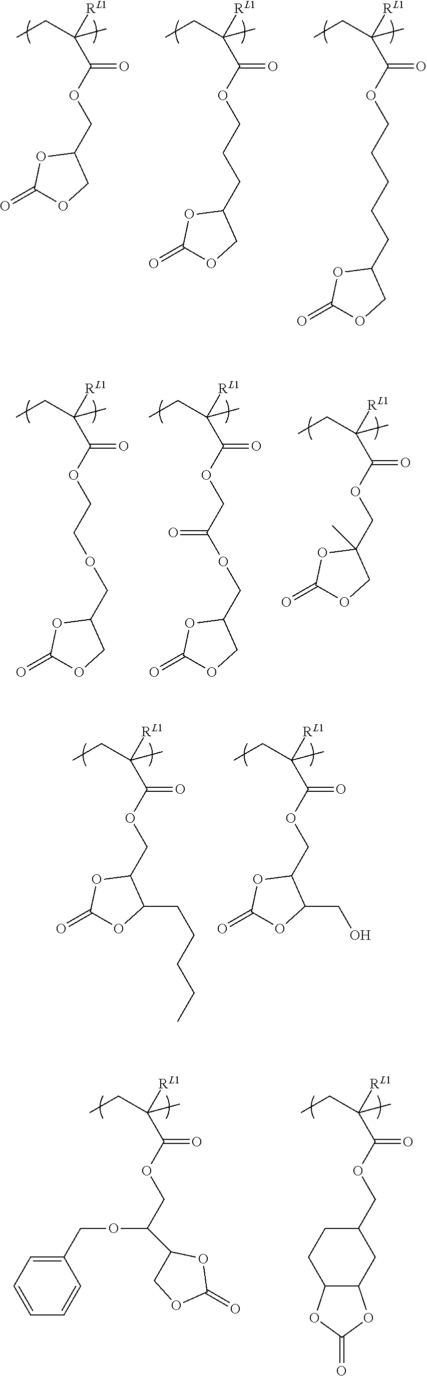

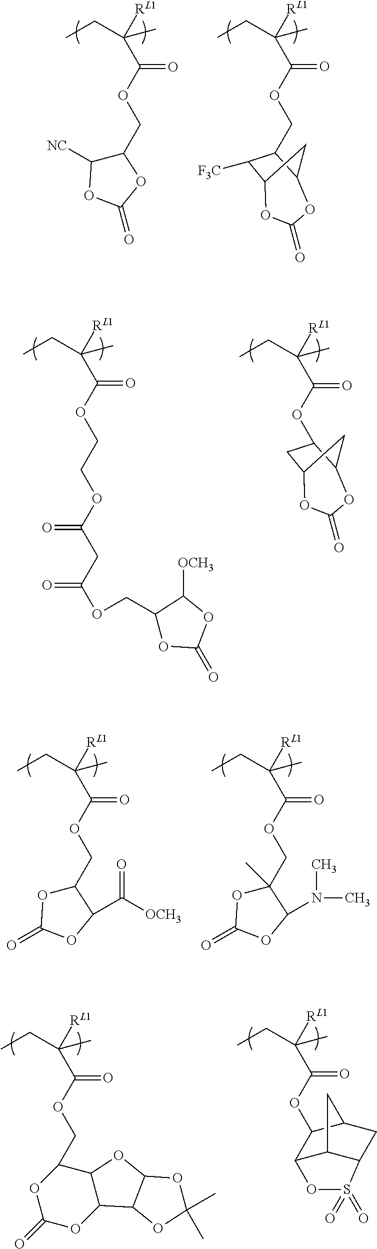

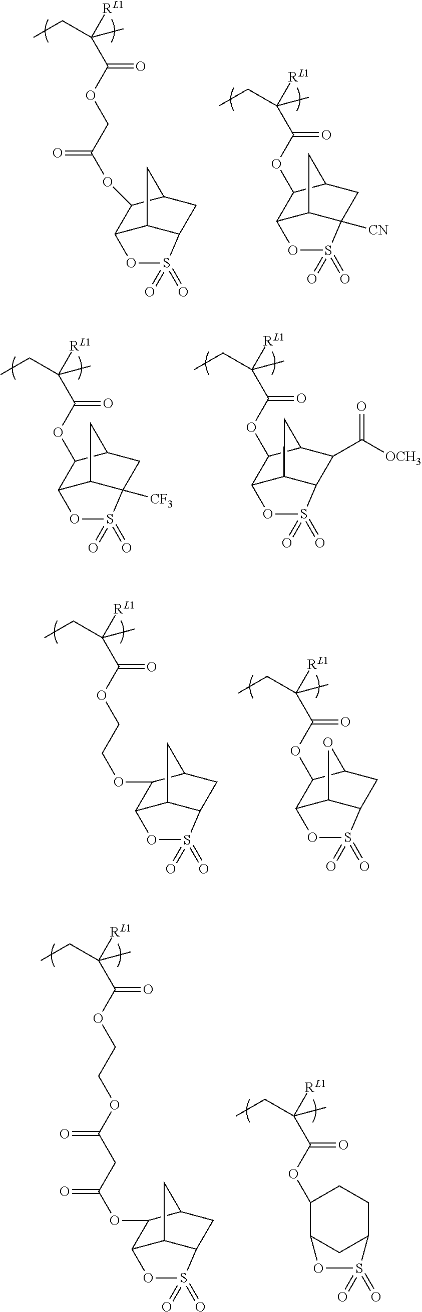

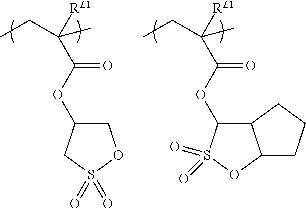

In each of the above formulae, R^{L1} represents a hydrogen atom, a fluorine atom, a methyl group, or a trifluoromethyl group.

The structural unit (II) is preferably a structural unit that includes a lactone structure or a cyclic carbonate structure.

In the case in which the polymer (A) has the structural unit (V), the lower limit of a proportion of the structural unit (V) contained with respect to the total structural units constituting the polymer (A) is preferably 1 mol %, more preferably 5 mol %, and still more preferably 10 mol %. The upper limit of the proportion is preferably 40 mol %, more preferably 35 mol %, and still more preferably 30 mol %.

### (B) Acid Generator

The acid generator (B) is a substance that generates an acid upon an exposure. The exposure light is exemplified by types of exposure light similar to those exemplified as the exposure light in the exposing step of the method of forming a resist pattern of an other embodiment of the present invention, described later, and the like. Due to the acid generated upon the exposure, the acid-labile group (a) in the structural unit (I) contained in the polymer (A) is dissociated to yield a carboxy group, whereby a difference in solubility of the resist film in the developer solution is generated between light-exposed regions and light-unexposed regions, and thus forming a resist pattern is enabled.

Examples of the acid generated from the acid generator (B) include sulfonic acid, imidic acid, and the like.

Regarding a form of the acid generator (B) contained in the radiation-sensitive resin composition, the form may be, for example: a low-molecular weight compound as described later (hereinafter, may be also referred to as “(B) acid generating agent” or “acid generating agent (B)”); a radiation-sensitive acid generating polymer (hereinafter, may be also referred to as “(B) acid generating polymer” or “acid generating polymer (B)”); or both of these forms. The “low-molecular weight compound” as referred to herein means a compound having a molecular weight of no greater than 1,000, without being accompanied by molecular weight distribution. The “radiation-sensitive acid generating polymer” as referred to herein means a polymer having a structural unit (the structural unit (VI) that generates an acid upon an exposure. In other words, the acid generating polymer (B) may also be deemed to be a form of the acid generator (B) incorporated as a part of the polymer. The acid generating polymer (B) may be a polymer that differs from the polymer (A) and polymer (B). The radiation-sensitive resin composition may contain one type, or two or more types of the acid generator (B).

(B) Acid Generating Agent

The acid generating agent (B) is exemplified by an onium salt compound, an N-sulfonyloxyimide compound, a sulfonimide compound, a halogen-containing compound, a diazo ketone compound, and the like.

Examples of the onium salt compound include a sulfonium salt, a tetrahydrothiophenium salt, an iodonium salt, a phosphonium salt, a diazonium salt, a pyridinium salt, and the like.

Specific examples of the acid generating agent (B) include compounds described in, for example, paragraphs [0080] to [0113] of Japanese Unexamined Patent Application, Publication No. 2009-134088, and the like.

Examples of the acid generating agent (B) which generates sulfonic acid upon an exposure include a compound (hereinafter, may be also referred to as “(B) compound” or “compound (B)”) represented by the following formula (4), and the like.

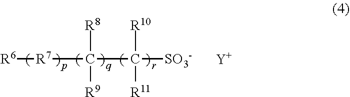

In the above formula (4): R⁶ represents a monovalent organic group having 1 to 30 carbon atoms; R⁷ represents a divalent linking group; R⁸ and R⁹ each independently represent a hydrogen atom or a monovalent hydrocarbon group having 1 to 20 carbon atoms; R¹⁰ and R¹¹ each independently represent a fluorine atom or a monovalent fluorinated hydrocarbon group having 1 to 20 carbon atoms; p is an integer of 0 to 10, q is an integer of 0 to 10, and r is an integer of 0 to 10, wherein a sum of p, q, and r is no less than 1 and no greater than 30, in a case in which p is no less than 2, a plurality of R⁷s are the same or different from each other, in a case in which q is no less than 2, a plurality of R⁸s are the same or different from each other and a plurality of R⁹s are the same or different from each other, and in a case in which r is no less than 2, a plurality of R¹⁰s are the same or different from each other and a plurality of R¹¹s are the same or different from each other; and Y⁺ represents a monovalent radiation-sensitive onium cation.

Examples of the monovalent organic group having 1 to 30 carbon atoms represented by R⁶ include groups similar to the groups exemplified as the monovalent organic group having 1 to 20 carbon atoms which may be represented by R², R³, or R⁴ in the above formula (1), and the like.

R⁶ is exemplified by a monovalent group including a ring structure having 5 or more ring atoms. Examples of the monovalent group including a ring structure having 5 or more ring atoms include a monovalent group that includes an alicyclic structure having 5 or more ring atoms, a monovalent group that includes an aliphatic heterocyclic structure having 5 or more ring atoms, a monovalent group that includes an aromatic carbocyclic structure having 5 or more ring atoms, a monovalent group that includes an aromatic heterocyclic structure having 5 or more ring atoms, and the like. These ring structures may have a substituent. Examples of the substituent include halogen atoms such as a fluorine atom and an iodine atom, a hydroxy group, a carboxy group, a cyano group, a nitro group, an alkoxy group, an alkoxycarbonyl group, an alkoxycarbonyloxy group, an acyl group, an acyloxy group, and the like.

Examples of the alicyclic structure having 5 or more ring atoms include:
monocyclic saturated alicyclic structures such as a cyclopentane structure, a cyclohexane structure, a cycloheptane structure, a cyclooctane structure, a cyclononane structure, a cyclodecane structure, and a cyclododecane structure;
monocyclic unsaturated alicyclic structures such as a cyclopentene structure, a cyclohexene structure, a cycloheptene structure, a cyclooctene structure, and a cyclodecene structure;
polycyclic saturated alicyclic structures such as a norbornane structure, an adamantane structure, a tricyclodecane structure, and a tetracyclododecane structure;
polycyclic unsaturated alicyclic structures such as a norbornene structure and a tricyclodecene structure;
structures having a steroid skeleton; and the like.

The “steroid skeleton” as referred to means a cyclopentanoperhydrophenanthrene nucleus, and means a skeleton resulting from condensation of three cyclohexane rings with one cyclopentane ring, or a skeleton in which one, or two or more carbon-carbon bonds of this skeleton is/are double bond(s).

Steroid skeletons are typically classified into the five types, i.e., cholestane structures, cholane structures, pregnane structures, androstane structures, and estrane structures. The steroid skeleton that gives R⁶ is preferably a cholane structure. In the case in which R⁶ represents the monovalent group having a structure that has a steroid skeleton as the ring structure having 5 or more ring atoms, R⁶ represents preferably a 3,7,12-trioxycholan-24-yl group, a 3,12-dihydroxycholan-24-yl group, or a 3,7,12-trihydroxycholan-24-yl group, and more preferably a 3,7,12-trioxycholan-24-yl group.

Examples of the aliphatic heterocyclic structure having 5 or more ring atoms include:
lactone structures such as a hexanolactone structure and a norbornanelactone structure;
sultone structures such as a hexanosultone structure and a norbornanesultone structure;
oxygen atom-containing heterocyclic structures such as an oxacycloheptane structure and an oxanorbornane structure;
nitrogen atom-containing heterocyclic structures such as an azacyclohexane structure and a diazabicyclooctane structure;
sulfur atom-containing heterocyclic structures such as a thiacyclohexane structure and a thianorbornane structure; and the like.

Examples of the aromatic carbocyclic structure having 5 or more ring atoms include a benzene structure, a naphthalene structure, a phenanthrene structure, an anthracene structure, and the like.

Examples of the aromatic heterocyclic structure having 5 or more ring atoms include:
oxygen atom-containing heterocyclic structures such as a furan structure, a pyran structure, a benzofuran structure, and a benzopyran structure;
nitrogen atom-containing heterocyclic structures such as a pyridine structure, a pyrimidine structure, and an indole structure; and the like.

Examples of the divalent linking group which may be represented by R⁷ include a carbonyl group, an ether group, a carbonyloxy group, an oxycarbonyl group, an oxycarbonyloxy group, a sulfide group, a thiocarbonyl group, a sulfonyl group, a divalent hydrocarbon group, a combination thereof, and the like. Of these, a carbonyloxy group, a sulfonyl group, an alkanediyl group, or a divalent alicyclic saturated hydrocarbon group is preferred, and a carbonyloxy group or a sulfonyl group is more preferred. It is to be noted that in the case in which p is no less than 2, the divalent linking group, being a group other than a divalent hydrocarbon group, is typically adjacent to only a divalent hydrocarbon group.

The monovalent hydrocarbon group having 1 to 20 carbon atoms which may be represented by R⁸ or R⁹ is exemplified by an alkyl group having 1 to 20 carbon atoms, and the like.

R⁸ and R⁹ each represent preferably a hydrogen atom or an alkyl group having 1 to 20 carbon atoms, and more preferably a hydrogen atom.

The monovalent fluorinated hydrocarbon group having 1 to 20 carbon atoms which may be represented by R¹⁰ or R¹¹ is exemplified by a group obtained by substituting with a fluorine atom at least one hydrogen atom contained in a monovalent hydrocarbon group having 1 to 20 carbon atoms, and the like.

R¹⁰ and R¹¹ each represent preferably a fluorine atom or a fluorinated alkyl group having 1 to 20 carbon atoms, and more preferably a fluorine atom.

p is preferably 0 to 5, more preferably 0 to 2, and still more preferably 0 or 1.

q is preferably 0 to 5, more preferably 0 to 2, and still more preferably 0 or 1.

The lower limit of r is preferably 1, and more preferably 2. When r is no less than 1, strength of the acid generated from the compound (B) can be increased. The upper limit of r is preferably 4, more preferably 3, and still more preferably 2.

The lower limit of the sum of p, q, and r is preferably 2, and more preferably 4. The upper limit of the sum of p, q, and r is preferably 20, and more preferably 10.

Examples of the monovalent radiation-sensitive onium cation represented by Y⁺ include monovalent cations (hereinafter, may be also referred to as “cations (r-a) to (r-c)”) represented by the following formulae (r-a) to (r-c), and the like.

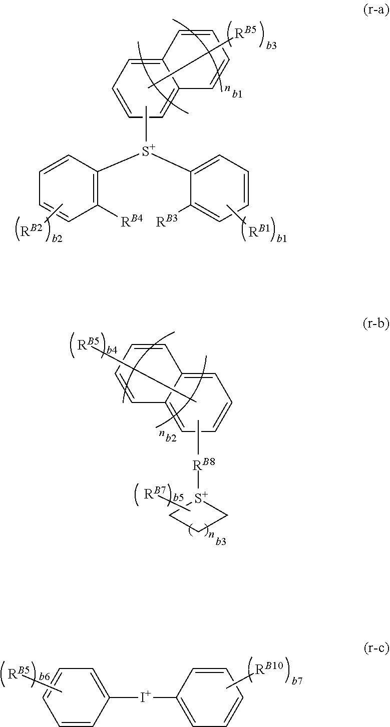

In the above formula (r-a), b1 is an integer of 0 to 4, wherein in a case in which b1 is 1, R^{B1} represents a halogen atom, a hydroxy group, a nitro group, or a monovalent organic group having 1 to 20 carbon atoms, and in a case in which b1 is no less than 2, a plurality of R^{B1}s are the same or different from each other, and each R^{B1} represents a halogen atom, a hydroxy group, a nitro group, or a monovalent organic group having 1 to 20 carbon atoms, or the plurality of R^{B1}s taken together represent a ring structure having 4 to 20 ring atoms together with the carbon chain to which the plurality of R^{B1}s bond; b2 is an integer of 0 to 4, wherein in a case in which b2 is 1, R^{B2} represents a halogen atom, a hydroxy group, a nitro group, or a monovalent organic group having 1 to 20 carbon atoms, and in a case in which b2 is no less than 2, a plurality of R^{B2}s are the same or different from each other, and each R^{B2} represents a halogen atom, a hydroxy group, a nitro group, or a monovalent organic group having 1 to 20 carbon atoms, or the plurality of R^{B2}s taken together represent a ring structure having 4 to 20 ring atoms together with the carbon chain to which the plurality of R^{B2}s bond; R^{B3} and R^{B4} each independently represent a hydrogen atom, a halogen atom, a hydroxy group, a nitro group, or a monovalent organic group having 1 to 20 carbon atoms, or R^{B3} and R^{B4} taken together represent a single bond; b3 is an integer of 0 to 11, wherein in a case in which b3 is 1, R^{B5} represents a halogen atom, a hydroxy group, a nitro group, or a monovalent organic group having 1 to 20 carbon atoms, and in a case in which b3 is no less than 2, a plurality of R^{B5}s are the same or different from each other, and each R^{B5} represents a halogen atom, a hydroxy group, a nitro group, or a monovalent organic group having 1 to 20 carbon atoms, or the plurality of R^{B5}s taken together represent a ring structure having 4 to 20 ring atoms together with the carbon chain to which the plurality of R^{B5}s bond; and n_{b1} is an integer of 0 to 3.

In the above formula (r-b), b4 is an integer of 0 to 9, wherein in a case in which b4 is 1, R^{B6} represents a halogen atom, a hydroxy group, a nitro group, or a monovalent organic group having 1 to 20 carbon atoms, and in a case in which b4 is no less than 2, a plurality of R^{B6}s are the same or different from each other, and each R^{B6} represents a halogen atom, a hydroxy group, a nitro group, or a monovalent organic group having 1 to 20 carbon atoms, or the plurality of R^{B6}s taken together represent a ring structure having 4 to 20 ring atoms together with the carbon chain to which the plurality of R^{B6}s bond; b5 is an integer of 0 to 10, wherein in a case in which b5 is 1, R^{B7} represents a halogen atom, a hydroxy group, a nitro group, or a monovalent organic group having 1 to 20 carbon atoms, and in a case in which b5 is no less than 2, a plurality of R^{B7}s are the same or different from each other, and each R^{B7} represents a halogen atom, a hydroxy group, a nitro group, or a monovalent organic group having 1 to 20 carbon atoms, or the plurality of R^{B7}s taken together represent a ring structure having 3 to 20 ring atoms together with the carbon atom or the carbon chain to which the plurality of R^{B7}s bond; n_{b3} is an integer of 0 to 3; R^{B8} represents a single bond or a divalent organic group having 1 to 20 carbon atoms; and n_{b2} is an integer of 0 to 2.

In the above formula (r-c), b6 is an integer of 0 to 5, wherein in a case in which b6 is 1, R^{B9} represents a halogen atom, a hydroxy group, a nitro group, or a monovalent organic group having 1 to 20 carbon atoms, and in a case in which b6 is no less than 2, a plurality of R^{B9}s are the same or different from each other, and each R^{B9} represents a halogen atom, a hydroxy group, a nitro group, or a monovalent organic group having 1 to 20 carbon atoms, or the plurality of R^{B9}s taken together represent a ring structure having 4 to 20 ring atoms together with the carbon chain to which the plurality of R^{B9}s bond; b7 is an integer of 0 to 5, wherein in a case in which b7 is 1, R^{B10} represents a halogen atom, a hydroxy group, a nitro group, or a monovalent organic group having 1 to 20 carbon atoms, and in a case in which b7 is no less than 2, a plurality of R^{B10}s are the same or different from each other, and each R^{B10} represents a halogen atom, a hydroxy group, a nitro group, or a monovalent organic group having 1 to 20 carbon atoms, or the plurality of R^{B10}s taken together represent a ring structure having 4 to 20 ring atoms together with the carbon chain to which the plurality of R^{B10}s bond.

Examples of the monovalent organic group having 1 to 20 carbon atoms which may be represented by R^{B1}, R^{B2}, R^{B3}, R^{B4}, R^{B5}, R^{B6}, R^{B7}, R^{B9} or R^{B10} include groups similar to the groups exemplified as the monovalent organic group having 1 to 20 carbon atoms which may be represented by R², R³, R⁴, or R⁵ in the above formula (1), and the like.

Examples of the divalent organic group which may be represented by R^{B8} include groups obtained by removing one hydrogen atom from the groups exemplified as the monovalent organic group having 1 to 20 carbon atoms which may be represented by R², R³, R⁴, or R⁵ in the above formula (1), and the like.

It is preferred that R^{B3} and R^{B4} each represent a hydrogen atom, or that R^{B3} and R^{B4} taken together represent a single bond.

b1 and b2 are each preferably 0 to 2, more preferably 0 or 1, and still more preferably 0. b3 is preferably 0 to 4, more preferably 0 to 2, and still more preferably 0 or 1. n_{b1} is preferably 0 or 1.

In the case in which b3 is no less than 1, R^{B5} represents preferably a cyclohexyl group or a cyclohexylsulfonyl group.

b6 and b7 are each preferably 0 to 3. In a case in which b6 and b7 are each no less than 1, R^{B9} and R^{B10} each represent preferably the hydrocarbon group, more preferably the chain hydrocarbon group, still more preferably the alkyl group, and yet more preferably an isopropyl group or a tert-butyl group.

The monovalent radiation-sensitive onium cation represented by Y⁺ is preferably the cation (r-a) or the cation (r-c).

Examples of the cation (r-a) include cations (hereinafter, may be also referred to as “cations (r-a-1) to (r-a-5)”) represented by the following formulae (r-a-1) to (r-a-5), and the like.

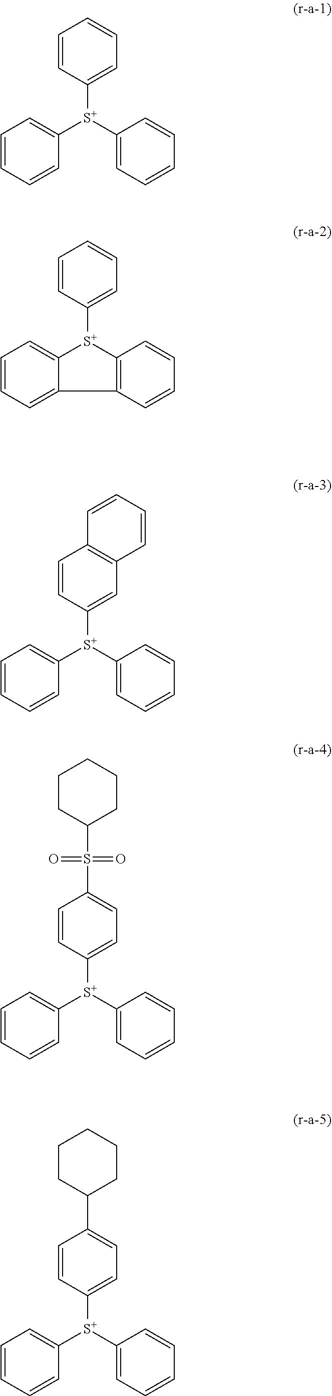 [SMILES: c1ccc([S+](c2ccccc2)c2ccccc2)cc1 ; c1ccc(-[s+]2c3ccccc3c3ccccc32)cc1 ; c1ccc([S+](c2ccccc2)c2ccc3ccccc3c2)cc1 ; O=S(=O)(c1ccc([S+](c2ccccc2)c2ccccc2)cc1)C1CCCCC1 ; c1ccc([S+](c2ccccc2)c2ccc(C3CCCCC3)cc2)cc1]

Examples of the cation (r-c) include cations (hereinafter, may be also referred to as “cations (r-c-1) to (r-c-4)”) represented by the following formulae (r-c-1) to (r-c-4), and the like.

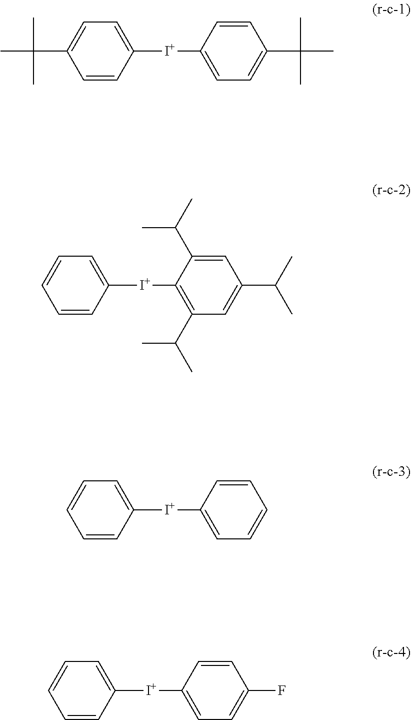 [SMILES: CC(C)(C)c1ccc([I+]c2ccc(C(C)(C)C)cc2)cc1 ; CC(C)c1cc(C(C)C)c([I+]c2ccccc2)c(C(C)C)c1 ; c1ccc([I+]c2ccccc2)cc1 ; Fc1ccc([I+]c2ccccc2)cc1]

Examples of the compound (B) include compounds (hereinafter, may be also referred to as “compounds (B1) to (B9)”) represented by the following formulae (4-1) to (4-9), and the like.

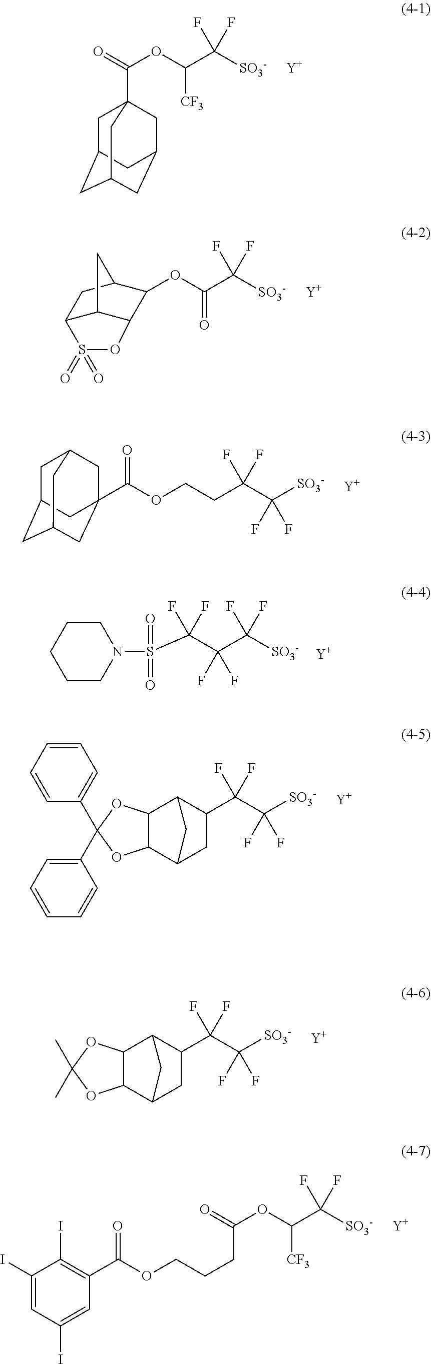

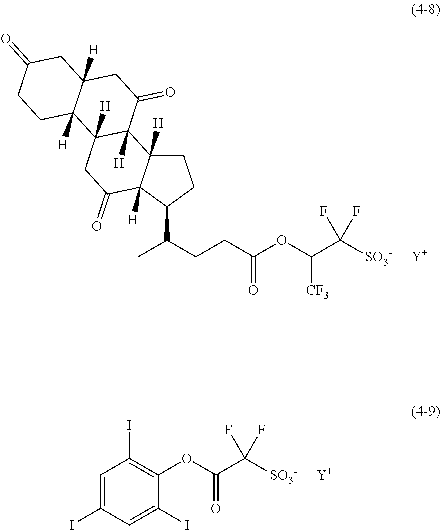

In the above formulae (4-1) to (4-9), Y⁺ is as defined in the above formula (4).

The lower limit of a content of the acid generating agent (B) in the radiation-sensitive resin composition with respect to 100 parts by mass of the polymer (A) is preferably 5 parts by mass, more preferably 10 parts by mass, and still more preferably 15 parts by mass. The upper limit of the content is preferably 60 parts by mass, more preferably 55 parts by mass, and still more preferably 50 parts by mass.

(B) Acid Generating Polymer

The acid generating polymer (B) is a polymer having a structural unit (the structural unit (VI)) that generates an acid upon an exposure. The structural unit (VI) is preferably, for example, a structural unit represented by the following formula (4′). It is to be noted that the structural unit (VI) may be included as a structural unit constituting the polymer (A) and/or may be included as a structural unit constituting a polymer other than the polymer (A), and is preferably included as a structural unit constituting the polymer (A). It is to be noted that in the case in which the polymer (A) has the structural unit (VI), the polymer (A) functions also as the acid generator (B).

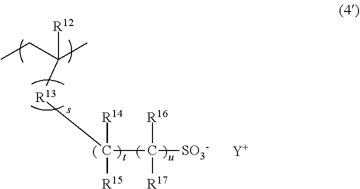

In the above formula (4′), R¹² represents a hydrogen atom, a fluorine atom, a methyl group, or a trifluoromethyl group; R¹³ represents a divalent linking group; R¹⁴ and R¹⁵ each independently represent a hydrogen atom or a monovalent hydrocarbon group having 1 to 20 carbon atoms; R¹⁶ and R¹⁷ each independently represent a fluorine atom or a monovalent fluorinated hydrocarbon group having 1 to 20 carbon atoms; s is an integer of 0 to 10, t is an integer of 0 to 10, and u is an integer of 0 to 10, wherein a sum of s, t, and u is no less than 1 and no greater than 30, in a case in which s is no less than 2, a plurality of R¹³s are the same or different from each other, in a case in which t is no less than 2, a plurality of R¹⁴s are the same or different from each other and a plurality of R¹⁵s are the same or different from each other, and in a case in which u is no less than 2, a plurality or R¹⁶s are the same or different from each other and a plurality of R¹⁷s are the same or different from each other, and Y⁺ represents a monovalent radiation-sensitive onium cation.

R¹² represents preferably a hydrogen atom or a methyl group, and more preferably a methyl group.

Examples of the divalent linking group represented by R¹³ include groups similar to those exemplified as the divalent linking group represented by R⁷ in the above formula (4), and the like. R¹³ represents preferably a carbonyloxy group.

Examples of the monovalent hydrocarbon group having 1 to 20 carbon atoms which may be represented by R¹⁴ or R¹⁵ include groups similar to the groups exemplified as the monovalent hydrocarbon group having 1 to 20 carbon atoms which may be represented by R′ or R⁹ in the above formula (4), and the like. R¹⁴ and R¹⁵ each represent preferably a hydrogen atom or an alkyl group having 1 to 20 carbon atoms, and more preferably a hydrogen atom.

Examples of the monovalent fluorinated hydrocarbon group having 1 to 20 carbon atoms which may be represented by R¹⁶ or R¹⁷ include groups similar to the groups exemplified as the monovalent hydrocarbon group having 1 to 20 carbon atoms which may be represented by R¹⁰ or R¹¹ in the above formula (4), and the like. R¹⁶ and R¹⁷ each represent preferably a fluorine atom or a fluorinated alkyl group having 1 to 20 carbon atoms, and more preferably a fluorine atom.

s is preferably 0 to 5, more preferably 0 or 2, and still more preferably 1.

t is preferably 0 to 5, more preferably 0 or 2, and still more preferably 0.

The lower limit of u is preferably 1. When u is no less than 1, strength of the acid generated from the structural unit represented by the above formula (4′) can be increased. The upper limit of u is preferably 4, more preferably 3, and still more preferably 2.

The lower limit of the sum of s, t, and u is preferably 2, and more preferably 3. The upper limit of the sum of s, t, and u is preferably 20, and more preferably 10.

The structural unit (VI) is preferably a structural unit (hereinafter, may be also referred to as “structural unit (VI-1)”) represented by the following formula (2′-1).

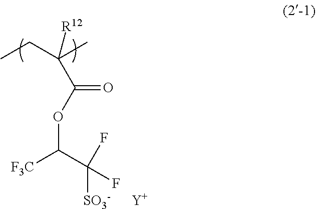

In the above formula (2′-1), R¹² and Y⁺ are as defined in the above formula (4′).

In the case in which the polymer (A) has the structural unit (VI), the lower limit of a proportion of the structural unit (IV) contained with respect to the total structural units constituting the polymer (A) is preferably 1 mol % and more preferably 5 mol %. On the other hand, the upper limit of the proportion of the structural unit with respect to the total structural units constituting the polymer (A) is preferably 20 mol %, and more preferably 15 mol %.

### (C) Acid Diffusion Control Agent

The acid diffusion control agent (C) is able to control a diffusion phenomenon in the resist film of the acid generated from the acid generator (B) and the like upon exposure, thereby exhibiting an effect of inhibiting unwanted chemical reactions in an unexposed region. Due to the acid diffusion control agent (C) being contained, the radiation-sensitive resin composition enables a resist pattern to be formed with favorable sensitivity to exposure light, superiority in LWR performance and CDU performance, and a broad process window. The radiation-sensitive resin composition may contain one, or two or more types of the acid diffusion control agent (C).

Examples of the acid diffusion control agent (C) include a nitrogen atom-containing compound, a compound (hereinafter, may be also referred to as “photodegradable base”) that is photosensitized by an exposure to generate a weak acid, and the like. The acid diffusion control agent (C) is preferably the photodegradable base. In this case, the sensitivity to exposure light, and the LWR performance, CDU performance, and process window can be further improved.

Examples of the nitrogen atom-containing compound include: amine compounds such as tripentylamine, trioctylamine, and tetrabutylammonium salicylate; amide group-containing compounds such as formamide and N,N-dimethylacetamide; urea compounds such as urea and 1,1-dimethylurea; nitrogen-containing heterocyclic compounds such as pyridine, 2,4,5-triphenylimidazole, N-(undecylcarbonyloxyethyl)morpholine, 1-(tert-butoxycarbonyl)-4-hydroxypiperidine, and N-t-pentyloxycarbonyl-4-hydroxypiperidine; and the like.

The photodegradable base is exemplified by a compound containing a radiation-sensitive onium cation and an anion of a weak acid; and the like. The photodegradable base generates an acid in light-exposed regions and increases solubility or insolubility of the polymer (A) in the developer solution, and consequently roughness of surfaces of the light-exposed regions after development is suppressed. On the other hand, the photodegradable base exerts a superior acid-capturing function by an anion in light-unexposed regions and serves as a quencher, and thus captures the acid diffused from the light-exposed regions. In other words, since the photodegradable base serves as a quencher only at the light-unexposed regions, the contrast resulting from a deprotection reaction is improved, and consequently the resolution can be improved.

Examples of the onium cation decomposable by the exposure include onium cations similar to those exemplified as the monovalent radiation-sensitive onium cation in the acid generating agent (B). Of these, a triphenylsulfonium cation, a phenyldibenzothiophenium cation, a diphenyliodonium cation, or a phenyl(4-fluorophenyl)iodonium cation is preferred.

Examples of the anion of the weak acid include anions represented by the following formulae, and the like.

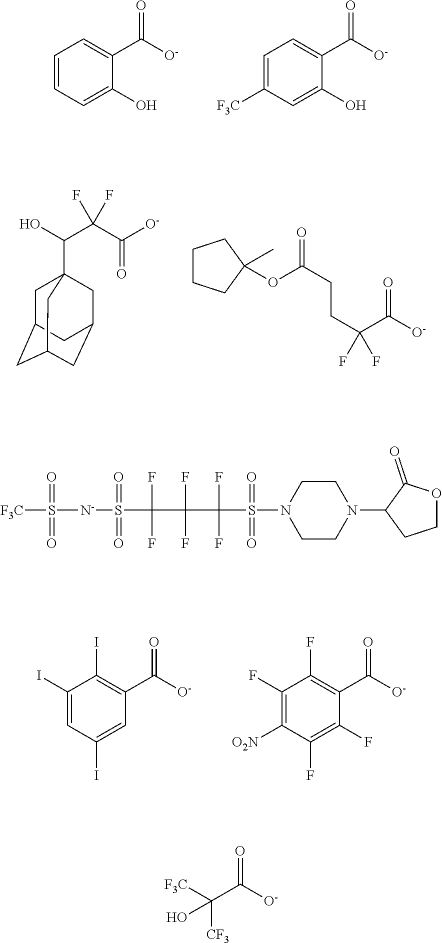

As the photodegradable base, a compound in which the onium cation decomposable by the exposure and the anion of the weak acid are appropriately combined can be used.

In the case of the radiation-sensitive resin composition containing the acid diffusion control agent (C), the lower limit of a proportion of the acid diffusion control agent (C) in the radiation-sensitive resin composition with respect to 100 mol % of the acid generating agent (B) is preferably 1 mol %, more preferably 5 mol %, and still more preferably 10 mol %. The upper limit of the proportion is preferably 100 mol %, more preferably 60 mol %, and still more preferably 50 mol %.

### (D) Organic Solvent

The radiation-sensitive resin composition typically contains the organic solvent (D). The organic solvent (D) is not particularly limited as long as it is a solvent capable of dissolving or dispersing at least the polymer (A) and the acid generator (B), as well as the acid diffusion control agent (C), and the other optional component(s), which is/are contained as needed.

The organic solvent (D) is exemplified by an alcohol solvent, an ether solvent, a ketone solvent, an amide solvent, an ester solvent, a hydrocarbon solvent, and the like. The radiation-sensitive resin composition may contain one, or two or more types of the organic solvent (D).

Examples of the alcohol solvent include:
aliphatic monohydric alcohol solvents having 1 to 18 carbon atoms such as 4-methyl-2-pentanol and n-hexanol;
alicyclic monohydric alcohol solvents having 3 to 18 carbon atoms such as cyclohexanol;
polyhydric alcohol solvents having 2 to 18 carbon atoms such as 1,2-propylene glycol;
polyhydric alcohol partial ether solvents having 3 to 19 carbon atoms such as propylene glycol 1-monomethyl ether; and the like.

Examples of the ether solvent include:
dialkyl ether solvents such as diethyl ether, dipropyl ether, dibutyl ether, dipentyl ether, diisoamyl ether, dihexyl ether, and diheptyl ether;
cyclic ether solvents such as tetrahydrofuran and tetrahydropyran;
aromatic ring-containing ether solvents such as diphenyl ether and anisole; and the like.

Examples of the ketone solvent include:
chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone;
cyclic ketone solvents such as cyclopentanone, cyclohexanone, cycloheptanone, cyclooctanone, and methylcyclohexanone;
2,4-pentanedione, acetonylacetone, and acetophenone; and the like.

Examples of the amide solvent include:
cyclic amide solvents such as N,N′-dimethylimidazolidinone and N-methylpyrrolidone;
chain amide solvents such as N-methylformamide, N,N-dimethylformamide, N,N-diethylformamide, acetamide, N-methylacetamide, N,N-dimethylacetamide, and N-methylpropionamide; and the like.

Examples of the ester solvent include:
monocarboxylic acid ester solvents such as n-butyl acetate and ethyl lactate;
lactone solvents such as y-butyrolactone and valerolactone;
polyhydric alcohol carboxylate solvents such as propylene glycol acetate;
polyhydric alcohol partial ether carboxylate solvents such as propylene glycol monomethyl ether acetate;
polyhydric carboxylic acid diester solvents such as diethyl oxalate;
carbonate solvents such as dimethyl carbonate and diethyl carbonate; and the like.

Examples of the hydrocarbon solvent include:
aliphatic hydrocarbon solvents having 5 to 12 carbon atoms such as n-pentane and n-hexane;
aromatic hydrocarbon solvents having 6 to 16 carbon atoms such as toluene and xylene; and the like.

The organic solvent (D) is preferably the alcohol solvent and/or the ester solvent, more preferably the polyhydric alcohol partial ether solvent having 3 to 19 carbon atoms and/or the polyhydric alcohol partial ether carboxylate solvent, and still more preferably propylene glycol 1-monomethyl ether and/or propylene glycol 1-monomethyl ether acetate.

In the case of the radiation-sensitive resin composition containing the organic solvent (D), the lower limit of a proportion of the organic solvent (D) with respect to the total components contained in the radiation-sensitive resin composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

Other Optional Component(s)

The other optional component(s) is/are exemplified by a surfactant and the like. The radiation-sensitive resin composition may contain one, or two or more types each of the other optional component(s).

### Method of Forming Resist Pattern

The method of forming a resist pattern according to the other embodiment of the present invention includes: a step (hereinafter, may be also referred to as “applying step”) of applying a radiation-sensitive resin composition directly or indirectly on a substrate; a step (hereinafter, may be also referred to as “exposing step”) of exposing a resist film formed by the applying step; and a step (hereinafter, may be also referred to as “developing step”) of developing the resist film exposed.

According to the method of forming a resist pattern, due to using the radiation-sensitive resin composition of the one embodiment of the present invention, described above, as the radiation-sensitive resin composition in the applying step, a resist pattern can be formed with favorable sensitivity to exposure light, superiority in LWR performance and CDU performance, and a broad process window.

Each step included in the method of forming a resist pattern is described below.

Applying Step

In this step, the radiation-sensitive resin composition is applied directly or indirectly on the substrate. By this step, the resist film is formed directly or indirectly on the substrate.

In this step, the radiation-sensitive resin composition of the one embodiment of the present invention, described above, is used as the radiation-sensitive resin composition.

The substrate is exemplified by a conventionally well-known substrate such as a silicon wafer, a wafer coated with silicon dioxide or aluminum, and the like. Furthermore, the substrate may be a substrate that has been subjected to a pretreatment, e.g., a hydrophobilization treatment such as a hexamethyldisilazane (hereinafter, may be also referred to as “HMDS”) treatment. In addition, the case of indirectly applying the radiation-sensitive resin composition on the substrate may be, for example, a case of applying the radiation-sensitive resin composition on an antireflective film formed on the substrate, and the like. Such an antireflective film is exemplified by an organic or inorganic antireflective film disclosed in, for example, Japanese Examined Patent Application, Publication No. H6-12452, Japanese Unexamined Patent Application, Publication No. S59-93448, and the like.

An application procedure is exemplified by spin coating, cast coating, roll coating, and the like. After the application, prebaking (hereinafter, may be also referred to as “PB”) may be carried out as needed for evaporating the solvent remaining in the coating film. The lower limit of a PB temperature is preferably 60° C., and more preferably 80° C. The upper limit of the PB temperature is preferably 150° C., and more preferably 140° C. The lower limit of a PB time period is preferably 5 sec, and more preferably 10 sec. The upper limit of the PB time period is preferably 600 sec, and more preferably 300 sec. The lower limit of an average thickness of the resist film formed is preferably 10 nm, and more preferably 20 nm. The upper limit of the average thickness is preferably 1,000 nm, and more preferably 500 nm.

Exposing Step

In this step, the resist film formed by the applying step is exposed. This exposure is carried out by irradiation with an exposure light through a photomask (as the case may be, through a liquid immersion medium such as water). Examples of the exposure light include: electromagnetic waves such as visible light rays, ultraviolet rays, far ultraviolet rays, extreme ultraviolet rays (EUV), X-rays, and 7-rays; charged particle rays such as electron beams and a-rays; and the like, which may be selected in accordance with a line width of the intended pattern, and the like. Of these, far ultraviolet rays, EUV, or electron beams are preferred; an ArF excimer laser beam (wavelength: 193 nm), a KrF excimer laser beam (wavelength: 248 nm), EUV (wavelength: 13.5 nm), or electron beams are more preferred; an ArF excimer laser beam, EUV, or electron beams are still more preferred; and EUV or electron beams are particularly preferred.

It is preferred that post exposure baking (hereinafter, may be also referred to as “PEB”) is carried out after the exposure to promote dissociation of the acid-labile group included in the polymer (A) etc., mediated by the acid generated from the acid generating agent (B), etc., upon the exposure in exposed regions of the resist film. This PEB enables an increase in a difference in solubility of the resist film in a developer solution between the light-exposed regions and light-unexposed regions. The lower limit of a temperature of the PEB is preferably 50° C., more preferably 80° C., and still more preferably 100° C. The upper limit of the temperature of the PEB is preferably 180° C., and more preferably 130° C. The lower limit of a time period of the PEB is preferably 5 sec, more preferably 10 sec, and still more preferably 30 sec. The upper limit of the time period of the PEB is preferably 600 sec, more preferably 300 sec, and still more preferably 100 sec.

Developing Step

In this step, the resist film exposed is developed. Accordingly, formation of a predetermined resist pattern is enabled. After the development, washing with a rinse agent such as water or an alcohol and then drying is typically performed. The development procedure in the developing step may be carried out by either development with an alkali, or development with an organic solvent.

In the case of the development with an alkali, the developer solution for use in the development is exemplified by alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (hereinafter, may be also referred to as “TMAH”), pyrrole, piperidine, choline, 1,8-diazabicyclo-[5.4.0]-7-undecene, and 1,5-diazabicyclo-[4.3.0]-5-nonene; and the like. Of these, an aqueous TMAH solution is preferred, and a 2.38% by mass aqueous TMAH solution is more preferred.

In the case of the development with an organic solvent, the developer solution is exemplified by: an organic solvent such as a hydrocarbon solvent, an ether solvent, an ester solvent, a ketone solvent, and an alcohol solvent; a solution containing the organic solvent; and the like. Exemplary organic solvents include one, or two or more types of solvents exemplified as the organic solvent (D) for the radiation-sensitive resin composition, and the like. Of these, the ester solvent or the ketone solvent is preferred. The ester solvent is preferably an acetic acid ester solvent, and more preferably n-butyl acetate. The ketone solvent is preferably a chain ketone, and more preferably 2-heptanone. The lower limit of the content of the organic solvent in the developer solution is preferably 80% by mass, more preferably 90% by mass, still more preferably 95% by mass, and particularly preferably 99% by mass. Components other than the organic solvent in the organic solvent developer solution are exemplified by water, silicone oil, and the like.

Examples of the development procedure include: a dipping procedure in which the substrate is immersed for a given time period in the developer solution charged in a container; a puddle procedure in which the developer solution is placed to form a dome-shaped bead by way of the surface tension on the surface of the substrate for a given time period to conduct a development; a spraying procedure in which the developer solution is sprayed onto the surface of the substrate; a dynamic dispensing procedure in which the developer solution is continuously applied onto the substrate, which is rotated at a constant speed, while scanning with a developer solution application nozzle at a constant speed; and the like.

The pattern to be formed according to the method of forming a resist pattern is exemplified by a line-and-space pattern, a hole pattern, and the like.

### Polymer

The polymer of still another embodiment of the present invention is described as the polymer (A) in the radiation-sensitive resin composition of the one embodiment of the present invention, described above. The polymer can be suitably used as a component of the radiation-sensitive resin composition.

### Compound

The compound of yet another embodiment of the present invention is a compound (hereinafter, may be also referred to as “(M) monomer” or “monomer (M)”) represented by the following formula (1′). The compound may be suitably used as a compound for synthesizing: the polymer (A) to be contained in the radiation-sensitive resin composition of the one embodiment of the present invention; or the polymer of the still another embodiment of the present invention.

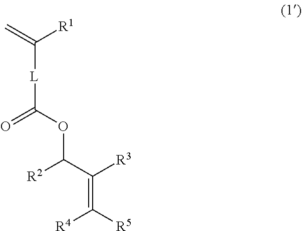

In the above formula (1′), R¹, R², R³, and R⁴ are as defined in the above formula (1).

The compound can be synthesized by, for example, a method described in EXAMPLES, described later, and the like.

### EXAMPLES

Hereinafter, the present invention is explained in detail by way of Examples, but the present invention is not in any way limited to the following Examples. Measuring methods for physical properties are each shown below.

Weight Average Molecular Weight (Mw), Number Average Molecular Weight (Mn), and Dispersity Index (Mw/Mn)

Measurements of the Mw and the Mn of the polymer (A) were carried out in accordance with the conditions described in the aforementioned paragraph “Method for Measuring Mw and Mn”. The dispersity index (Mw/Mn) of the polymer was calculated from the measurement results of the Mw and the Mn.

### Synthesis of Monomer (M)

Compounds (hereinafter, may be also referred to as “monomers (M-1) to (M-30)”) represented by the following formulae (M-1) to (M-30) as the monomers (M) were synthesized in accordance with the following method.

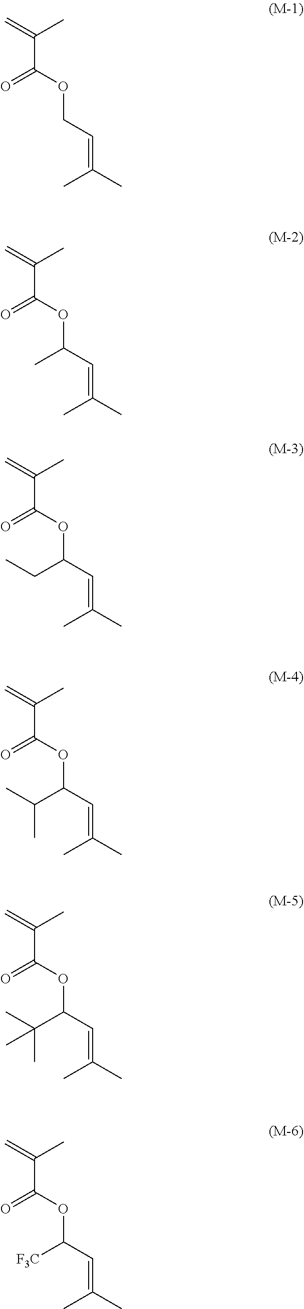

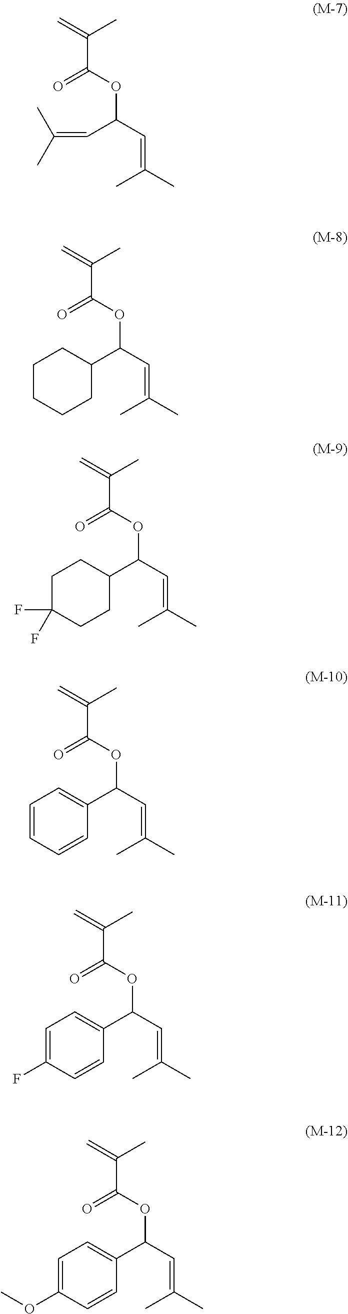

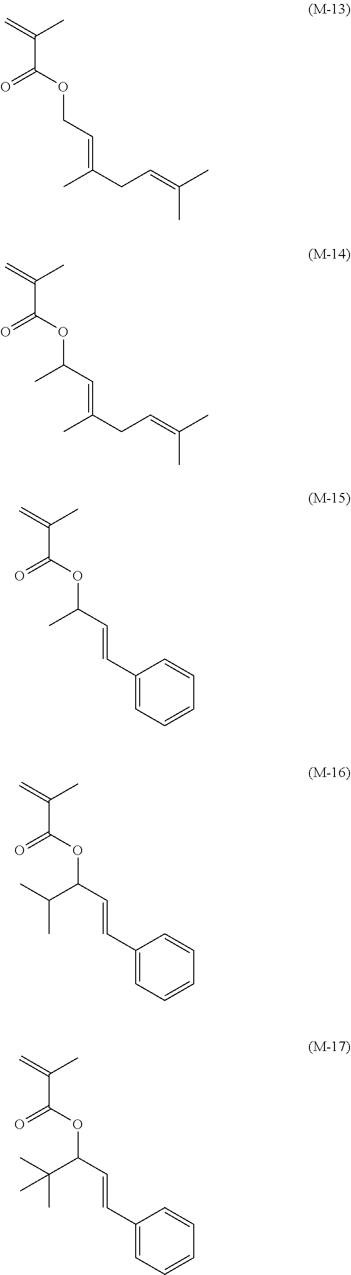 [SMILES: C=C(C)C(=O)OC/C=C(\C)CC=C(C)C ; C=C(C)C(=O)OC(C)/C=C(\C)CC=C(C)C ; C=C(C)C(=O)OC(C)/C=C/c1ccccc1 ; C=C(C)C(=O)OC(/C=C/c1ccccc1)C(C)C ; C=C(C)C(=O)OC(/C=C/c1ccccc1)C(C)(C)C]

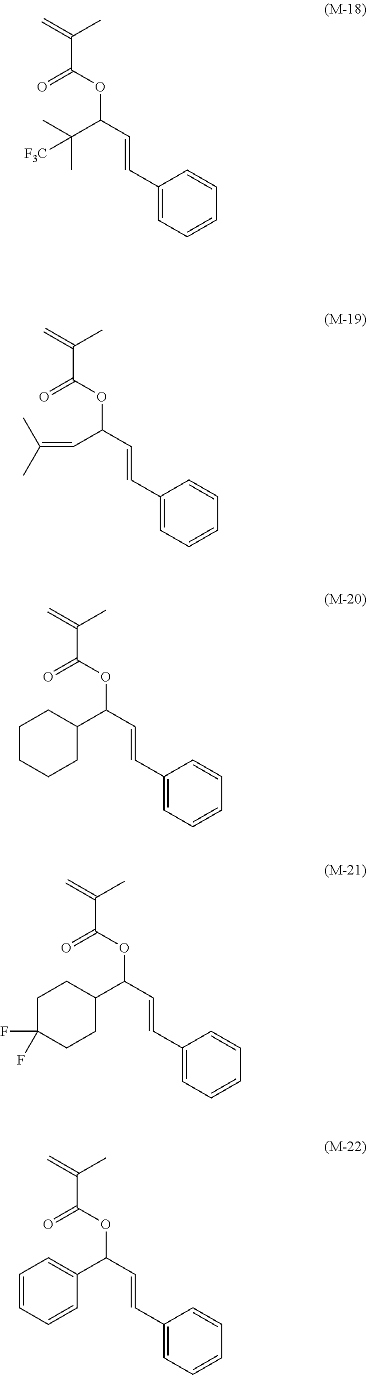 [SMILES: C=C(C)C(=O)OC(/C=C/c1ccccc1)C(C)(C)C(F)(F)F ; C=C(C)C(=O)OC(C=C(C)C)/C=C/c1ccccc1 ; C=C(C)C(=O)OC(/C=C/c1ccccc1)C1CCCCC1 ; C=C(C)C(=O)OC(/C=C/c1ccccc1)C1CCC(F)(F)CC1 ; C=C(C)C(=O)OC(/C=C/c1ccccc1)c1ccccc1]

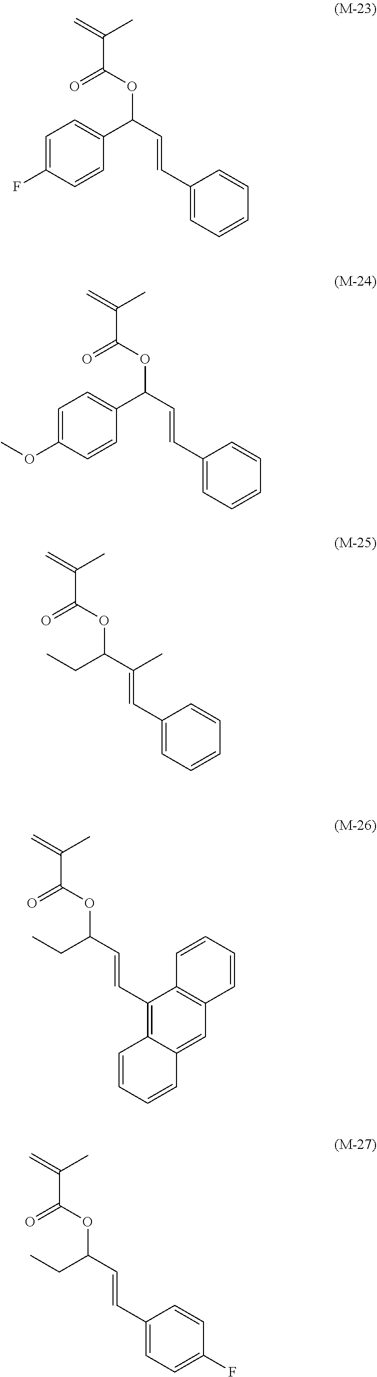 [SMILES: C=C(C)C(=O)OC(/C=C/c1ccccc1)c1ccc(F)cc1 ; C=C(C)C(=O)OC(/C=C/c1ccccc1)c1ccc(OC)cc1 ; C=C(C)C(=O)OC(CC)/C(C)=C/c1ccccc1 ; C=C(C)C(=O)OC(/C=C/c1c2ccccc2cc2ccccc12)CC ; C=C(C)C(=O)OC(/C=C/c1ccc(F)cc1)CC]

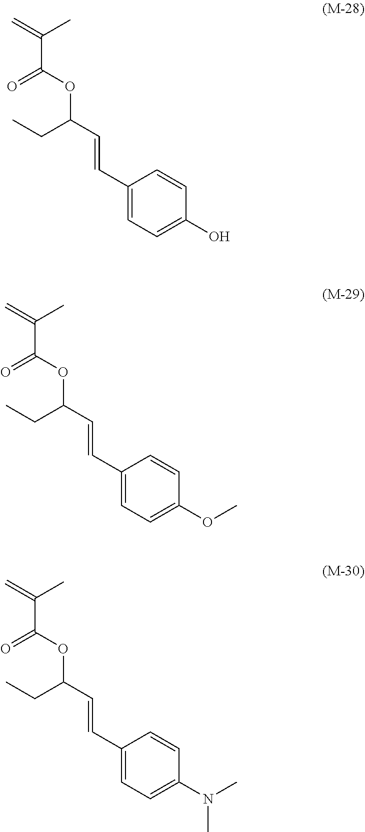 [SMILES: C=C(C)C(=O)OC(/C=C/c1ccc(O)cc1)CC ; C=C(C)C(=O)OC(/C=C/c1ccc(OC)cc1)CC ; C=C(C)C(=O)OC(/C=C/c1ccc(N(C)C)cc1)CC]

### Synthesis Example 1-1: Synthesis of Monomer (M-1)

Into a 3 L eggplant-shaped flask were weighed 200 g of prenol and 282 g of triethylamine, which were then dissolved in 1,500 mL of dichloromethane. The solvent was cooled to 0° C., and 243 g of methacryloyl chloride was added dropwise thereto at such a rate that a solution temperature did not exceed 25° C. After completion of the dropwise addition, stirring was performed for 1 hour at 25° C. After completion of the reaction, an aqueous saturated ammonium chloride solution was added thereto and extraction with methylene chloride was conducted, followed by concentration in vacuo. A residue thus obtained was subjected to purification by column chromatography, whereby 233 g of the monomer (M-1) (yield percentage: 65%) was obtained. A synthesis scheme of the monomer (M-1) is shown below.

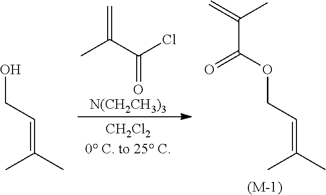

### Synthesis Examples 1-2 to 1-30: Syntheses of Monomers (M-2) to (M-30)

Monomers (M-2) to (M-30) were synthesized similarly to Synthesis Example 1-1, except for appropriately changing each precursor.

Synthesis of Polymer (A)

For syntheses of the polymers (A), compounds (hereinafter, may be also referred to as “monomers (M-31) to (M-60)”) represented by the following formulae (M-31) to (M-60) were used as monomers other than the monomers (M). It is to be noted that in the following Synthesis Examples, unless otherwise specified particularly, the term “parts by mass” means a value, provided that the total mass of the monomers used was 100 parts by mass, and the term “mol %” means a value, provided that the total mol number of the monomers used was 100 mol %.

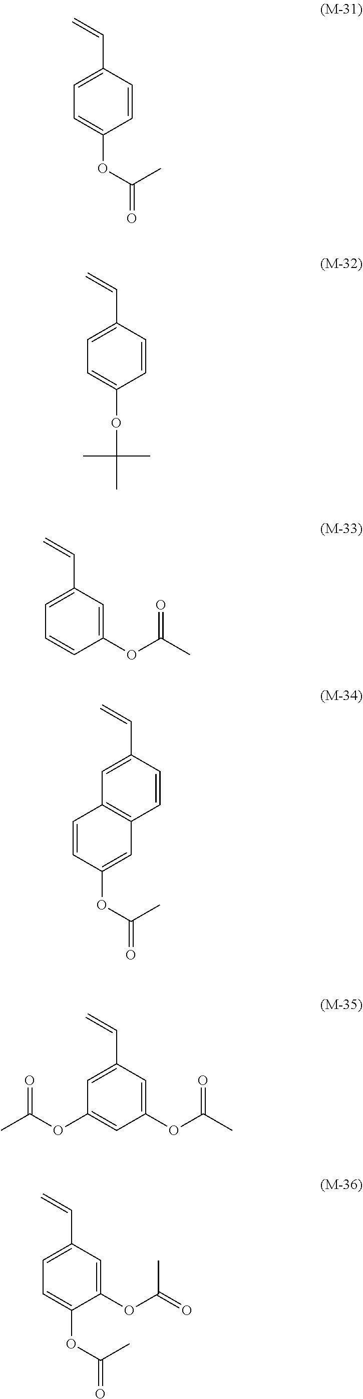

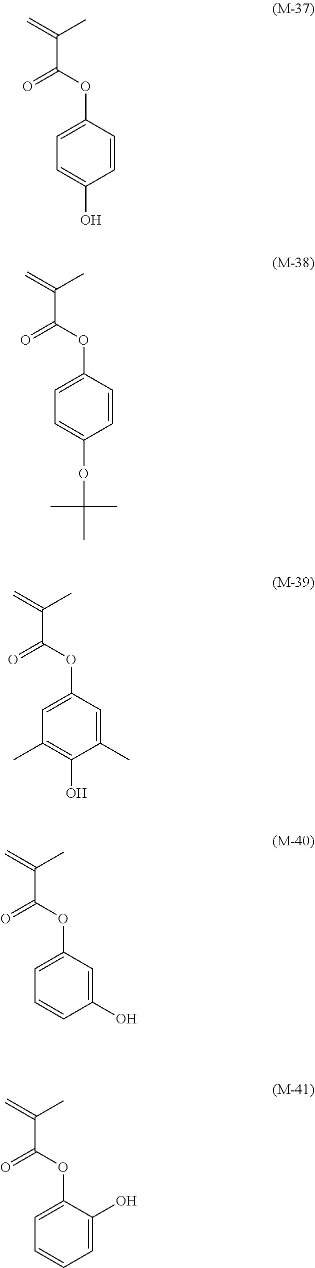 [SMILES: C=C(C)C(=O)Oc1ccc(O)cc1 ; C=C(C)C(=O)Oc1ccc(OC(C)(C)C)cc1 ; C=C(C)C(=O)Oc1cc(C)c(O)c(C)c1 ; C=C(C)C(=O)Oc1cccc(O)c1 ; C=C(C)C(=O)Oc1ccccc1O]

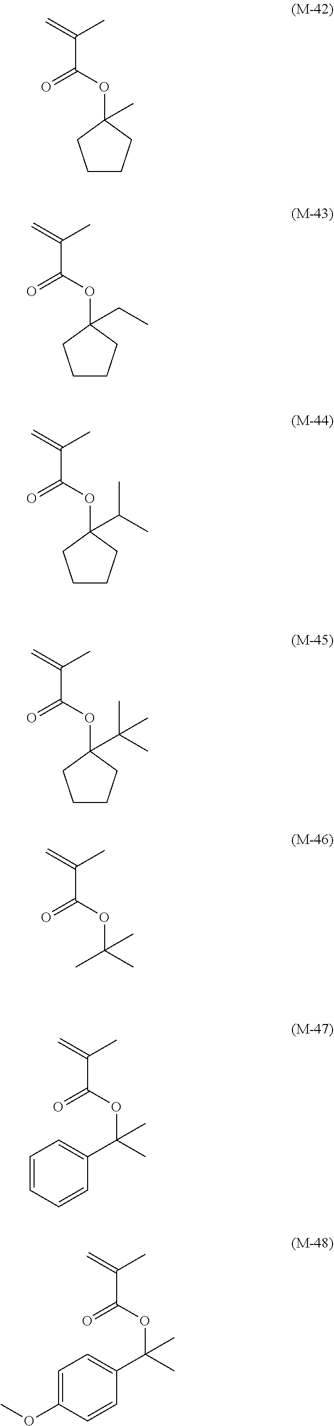

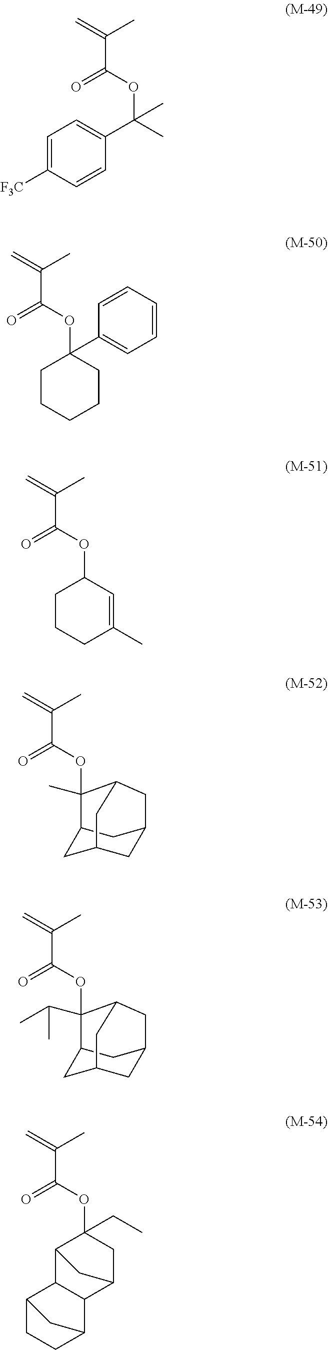

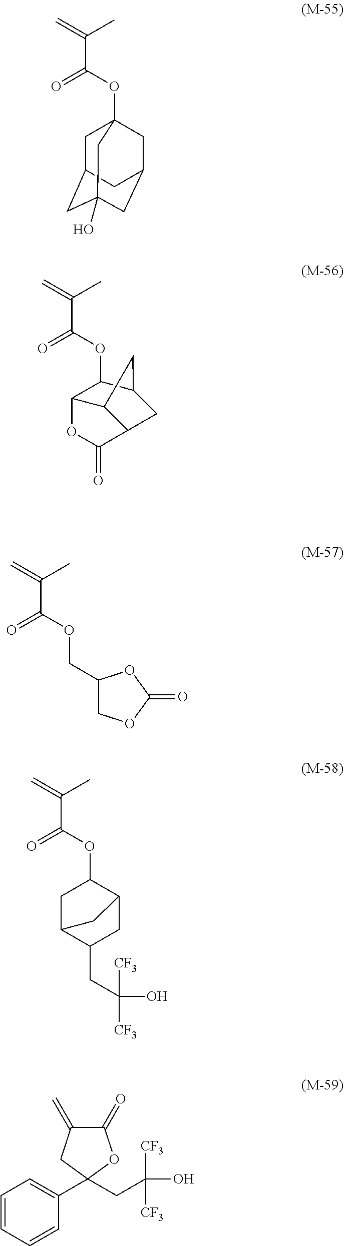 [SMILES: C=C(C)C(=O)OC12CC3CC(CC(O)(C3)C1)C2 ; C=C(C)C(=O)OC1C2CC3C(=O)OC1C3C2 ; C=C(C)C(=O)OCC1COC(=O)O1 ; C=C(C)C(=O)OC1CC2CC1CC2CC(O)(C(F)(F)F)C(F)(F)F ; C=C1CC(CC(O)(C(F)(F)F)C(F)(F)F)(c2ccccc2)OC1=O]

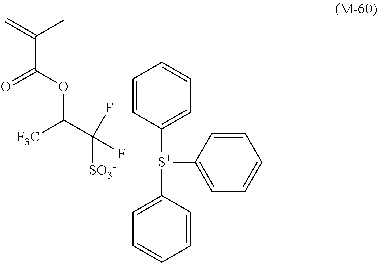

### Synthesis Example 2-1: Synthesis of Polymer (A-1)

The monomer (M-1) and the monomer (M-31) were dissolved in propylene glycol 1-monomethyl ether (200 parts by mass) such that the molar ratio became 40/60. Next, 6 mol % azobisisobutyronitrile (AIBN) was added as a polymerization initiator to prepare a monomer solution. Meanwhile, propylene glycol 1-monomethyl ether (100 parts by mass) was charged into an empty reaction vessel and heated to 85° C. with stirring. Next, the monomer solution prepared as described above was added dropwise over 3 hrs, followed by further heating at 85° C. for 3 hrs, whereby the polymerization reaction was performed for 6 hrs in total. After completion of the polymerization reaction, the polymerization solution was cooled to room temperature.

The cooled polymerization solution was charged into hexane (500 parts by mass with respect to the polymerization solution), and a thus precipitated white powder was filtered off. The white powder obtained by the filtration was washed twice with 100 parts by mass of hexane with respect to the polymerization solution, followed by filtering off and dissolution in propylene glycol 1-monomethyl ether (300 parts by mass). Next, methanol (500 parts by mass), triethylamine (50 parts by mass), and ultra-pure water (10 parts by mass) were added to a resulting solution, and a hydrolysis reaction was performed at 70° C. for 6 hrs with stirring. After completion of the hydrolysis reaction, the remaining solvent was distilled away and a solid thus obtained was dissolved in acetone (100 parts by mass). The solution was added dropwise into 500 parts by mass of water to permit coagulation of the resin, a solid thus obtained was filtered off, and drying at 50° C. for 12 hrs gave a white powdery polymer (A-1). With regard to the polymer (A-1) obtained, the Mw was 7,600, and the Mw/Mn was 1.55.

### Synthesis Examples 2-2 to 2-60 and 2-67 to 2-69: Syntheses of Polymers (A-2) to (A-60) and (a-1) to (a-3)

Polymers (A-2) to (A-60) and (a-1) to (a-3) were synthesized by a similar operation to that of Synthesis Example 2-1, except that each monomer of the type and in the usage proportion shown in Table 1 below was used.

### Synthesis Examples 2-61 to 2-65: Syntheses of Polymers (A-61) to (A-65)

Polymers (A-61) to (A-65) were synthesized by a similar operation to that of Synthesis Example 2-1, except that each monomer of the type and in the usage proportion shown in Table 1 below was used, and that the usage amount of each polymerization initiator was changed appropriately. The polymers (A-61) to (A-65) are polymers that have the same monomer formulation as the polymer (A-2), and for which the Mw and Mw/Mn are different.

### Synthesis Example 2-66: Synthesis of Polymer (A-66)

The monomer (M-1), the monomer (M-31), and the monomer (M-60) were dissolved in 2-butanone (200 parts by mass) such that the molar ratio became 40/50/10. Next, 6 mol % AIBN was added as a polymerization initiator to prepare a monomer solution. Meanwhile, 2-butanone (100 parts by mass) was charged into an empty reaction vessel and heated to 80° C. with stirring. Next, the monomer solution prepared as described above was added dropwise over 3 hrs, followed by further heating at 85° C. for 3 hrs, whereby the polymerization reaction was performed for 6 hrs in total. After completion of the polymerization reaction, the polymerization solution was cooled to room temperature.

The cooled polymerization solution was charged into methanol (2,000 parts by mass with respect to the polymerization solution), and a thus precipitated white powder was filtered off. A solid thus obtained was dissolved in acetone (100 parts by mass). The solution was added dropwise into 500 parts by mass of water to permit coagulation of the resin, and a solid thus obtained was filtered off. Drying at 50° C. for 12 hrs gave a white powdery polymer (A-66). The Mw of the polymer (A-66) obtained was 8,500, and the Mw/Mn was 1.86.

### Synthesis Example 2-70: Synthesis of Polymer (a-4)

Polymer (a-4) was synthesized by a similar operation to that of Synthesis Example 2-66 except that each monomer of the type and in the usage proportion shown in Table 1 below was used.

Types and usage proportions of the monomers that give each structural unit in the polymers (A) obtained in Synthesis Examples 2-1 to 2-70, as well as the Mw and the Mw/Mn thereof, are shown in Table 1 below. It is to be noted that in Table 1, “-” indicates that the corresponding monomer was not used.

TABLE 1

| | | Monomer that | | Monomer that | | Monomer that | | Monomer that | | | |
|---|---|---|---|---|---|---|---|---|---|---|---|
| | | gives structural | | gives structural | | gives structural | | gives other | | | |
| | | unit (I) | | unit (II) | | unit (III) | | structural unit | | | |
| | | | usage | | usage | | usage | | usage | | |
| | | | proportion | | proportion | | proportion | | proportion | Physical | |
| | (A) | | (% by | | (% by | | (% by | | (% by | property values | |
| | Polymer | type | mole) | type | mole) | type | mole) | type | mole) | Mw | Mw/Mn |
| Synthesis Example 2-1 | A-1 | M-1 | 40 | M-31 | 60 | — | — | — | — | 7,600 | 1.55 |
| Synthesis Example 2-2 | A-2 | M-1 | 50 | M-31 | 50 | — | — | — | — | 7,300 | 1.47 |
| Synthesis Example 2-3 | A-3 | M-1 | 60 | M-31 | 40 | — | — | — | — | 7,700 | 1.53 |
| Synthesis Example 2-4 | A-4 | M-2 | 50 | M-31 | 50 | — | — | — | — | 6,900 | 1.53 |
| Synthesis Example 2-5 | A-5 | M-3 | 50 | M-31 | 50 | — | — | — | — | 7,100 | 1.55 |
| Synthesis Example 2-6 | A-6 | M-4 | 50 | M-31 | 50 | — | — | — | — | 7,600 | 1.58 |
| Synthesis Example 2-7 | A-7 | M-5 | 50 | M-31 | 50 | — | — | — | — | 7,900 | 1.48 |
| Synthesis Example 2-8 | A-8 | M-6 | 50 | M-31 | 50 | — | — | — | — | 8,100 | 1.51 |
| Synthesis Example 2-9 | A-9 | M-7 | 50 | M-31 | 50 | — | — | — | — | 6,800 | 1.59 |
| Synthesis Example 2-10 | A-10 | M-8 | 50 | M-31 | 50 | — | — | — | — | 7,000 | 1.61 |
| Synthesis Example 2-11 | A-11 | M-9 | 50 | M-31 | 50 | — | — | — | — | 7,100 | 1.58 |
| Synthesis Example 2-12 | A-12 | M-10 | 50 | M-31 | 50 | — | — | — | — | 7,600 | 1.55 |
| Synthesis Example 2-13 | A-13 | M-11 | 50 | M-31 | 50 | — | — | — | — | 8,100 | 1.63 |
| Synthesis Example 2-14 | A-14 | M-12 | 50 | M-31 | 50 | — | — | — | — | 6,600 | 1.47 |
| Synthesis Example 2-15 | A-15 | M-13 | 50 | M-31 | 50 | — | — | — | — | 6,900 | 1.52 |
| Synthesis Example 2-16 | A-16 | M-14 | 50 | M-31 | 50 | — | — | — | — | 7,200 | 1.59 |
| Synthesis Example 2-17 | A-17 | M-15 | 50 | M-31 | 50 | — | — | — | — | 7,800 | 1.66 |
| Synthesis Example 2-18 | A-18 | M-16 | 50 | M-31 | 50 | — | — | — | — | 6,900 | 1.53 |
| Synthesis Example 2-19 | A-19 | M-17 | 50 | M-31 | 50 | — | — | — | — | 7,500 | 1.42 |
| Synthesis Example 2-20 | A-20 | M-18 | 50 | M-31 | 50 | — | — | — | — | 7,200 | 1.45 |
| Synthesis Example 2-21 | A-21 | M-19 | 50 | M-31 | 50 | — | — | — | — | 7,400 | 1.44 |
| Synthesis Example 2-22 | A-22 | M-20 | 50 | M-31 | 50 | — | — | — | — | 7,100 | 1.44 |
| Synthesis Example 2-23 | A-23 | M-21 | 50 | M-31 | 50 | — | — | — | — | 6,900 | 1.65 |
| Synthesis Example 2-24 | A-24 | M-22 | 50 | M-31 | 50 | — | — | — | — | 8,000 | 1.61 |
| Synthesis Example 2-25 | A-25 | M-23 | 50 | M-31 | 50 | — | — | — | — | 7,700 | 1.56 |
| Synthesis Example 2-26 | A-26 | M-24 | 50 | M-31 | 50 | — | — | — | — | 7,500 | 1.58 |
| Synthesis Example 2-27 | A-27 | M-25 | 50 | M-31 | 50 | — | — | — | — | 7,100 | 1.54 |
| Synthesis Example 2-28 | A-28 | M-26 | 50 | M-31 | 50 | — | — | — | — | 7,700 | 1.56 |
| Synthesis Example 2-29 | A-29 | M-27 | 50 | M-31 | 50 | — | — | — | — | 7,600 | 1.60 |
| Synthesis Example 2-30 | A-30 | M-28 | 50 | M-31 | 50 | — | — | — | — | 7,200 | 1.53 |
| Synthesis Example 2-31 | A-31 | M-29 | 50 | M-31 | 50 | — | — | — | — | 7,400 | 1.50 |
| Synthesis Example 2-32 | A-32 | M-30 | 50 | M-31 | 50 | — | — | — | — | 7,900 | 1.52 |
| Synthesis Example 2-33 | A-33 | M-1 | 25 | M-31/M-32 | 50/25 | — | — | — | — | 6,500 | 1.66 |
| Synthesis Example 2-34 | A-34 | M-1 | 50 | M-31/M-33 | 25/25 | — | — | — | — | 7,600 | 1.54 |
| Synthesis Example 2-35 | A-35 | M-1 | 50 | M-31/M-34 | 25/25 | — | — | — | — | 7,100 | 1.48 |
| Synthesis Example 2-36 | A-36 | M-1 | 50 | M-31/M-35 | 25/25 | — | — | — | — | 7,700 | 1.47 |
| Synthesis Example 2-37 | A-37 | M-1 | 50 | M-31/M-36 | 25/25 | — | — | — | — | 6,600 | 1.44 |
| Synthesis Example 2-38 | A-38 | M-1 | 50 | M-31/M-37 | 25/25 | — | — | — | — | 8,100 | 1.65 |
| Synthesis Example 2-39 | A-39 | M-1 | 25 | M-31/M-38 | 50/25 | — | — | — | — | 7,200 | 1.63 |
| Synthesis Example 2-40 | A-40 | M-1 | 50 | M-31/M-39 | 25/25 | — | — | — | — | 7,400 | 1.58 |
| Synthesis Example 2-41 | A-41 | M-1 | 50 | M-31/M-40 | 25/25 | — | — | — | — | 6,900 | 1.56 |
| Synthesis Example 2-42 | A-42 | M-1 | 50 | M-31/M-41 | 25/25 | — | — | — | — | 8,200 | 1.59 |
| Synthesis Example 2-43 | A-43 | M-1 | 25 | M-31 | 50 | M-42 | 25 | — | — | 7,400 | 1.54 |
| Synthesis Example 2-44 | A-44 | M-1 | 25 | M-31 | 50 | M-43 | 25 | — | — | 7,700 | 1.61 |
| Synthesis Example 2-45 | A-45 | M-1 | 25 | M-31 | 50 | M-44 | 25 | — | — | 8,300 | 1.64 |
| Synthesis Example 2-46 | A-46 | M-1 | 25 | M-31 | 50 | M-45 | 25 | — | — | 7,400 | 1.58 |
| Synthesis Example 2-47 | A-47 | M-1 | 25 | M-31 | 50 | M-46 | 25 | — | — | 7,100 | 1.63 |
| Synthesis Example 2-48 | A-48 | M-1 | 25 | M-31 | 50 | M-47 | 25 | — | — | 6,600 | 1.52 |
| Synthesis Example 2-49 | A-49 | M-1 | 25 | M-31 | 50 | M-48 | 25 | — | — | 6,800 | 1.53 |
| Synthesis Example 2-50 | A-50 | M-1 | 25 | M-31 | 50 | M-49 | 25 | — | — | 8,000 | 1.64 |
| Synthesis Example 2-51 | A-51 | M-1 | 25 | M-31 | 50 | M-50 | 25 | — | — | 7,800 | 1.58 |
| Synthesis Example 2-52 | A-52 | M-1 | 25 | M-31 | 50 | M-51 | 25 | — | — | 7,200 | 1.49 |
| Synthesis Example 2-53 | A-53 | M-1 | 35 | M-31 | 45 | M-52 | 20 | — | — | 7,300 | 1.53 |
| Synthesis Example 2-54 | A-54 | M-1 | 35 | M-31 | 45 | M-53 | 20 | — | — | 7,500 | 1.51 |
| Synthesis Example 2-55 | A-55 | M-1 | 35 | M-31 | 45 | M-54 | 20 | — | — | 8,200 | 1.44 |
| Synthesis Example 2-56 | A-56 | M-1 | 35 | M-31 | 45 | — | — | M-55 | 20 | 8,100 | 1.46 |
| Synthesis Example 2-57 | A-57 | M-1 | 35 | M-31 | 45 | — | — | M-56 | 20 | 7,700 | 1.49 |
| Synthesis Example 2-58 | A-58 | M-1 | 35 | M-31 | 45 | — | — | M-57 | 20 | 7,100 | 1.55 |
| Synthesis Example 2-59 | A-59 | M-1 | 35 | M-31 | 45 | — | — | M-58 | 20 | 7,600 | 1.49 |
| Synthesis Example 2-60 | A-60 | M-1 | 35 | M-31 | 45 | — | — | M-59 | 20 | 7,900 | 1.46 |
| Synthesis Example 2-61 | A-61 | M-1 | 50 | M-31 | 50 | — | — | — | — | 19,500 | 1.65 |
| Synthesis Example 2-62 | A-62 | M-1 | 50 | M-31 | 50 | — | — | — | — | 13,200 | 1.65 |
| Synthesis Example 2-63 | A-63 | M-1 | 50 | M-31 | 50 | — | — | — | — | 9,500 | 1.62 |
| Synthesis Example 2-64 | A-64 | M-1 | 50 | M-31 | 50 | — | — | — | — | 5,600 | 1.48 |
| Synthesis Example 2-65 | A-65 | M-1 | 50 | M-31 | 50 | — | — | — | — | 3,600 | 1.57 |
| Synthesis Example 2-66 | A-66 | M-1 | 40 | M-31 | 50 | — | — | M-60 | 10 | 8,500 | 1.86 |
| Synthesis Example 2-67 | a-1 | — | — | M-31 | 50 | M-42 | 50 | — | — | 6,500 | 1.32 |
| Synthesis Example 2-68 | a-2 | — | — | M-31 | 50 | M-43 | 50 | — | — | 7,100 | 1.37 |
| Synthesis Example 2-69 | a-3 | — | — | M-31 | 50 | M-44 | 50 | — | — | 7,600 | 1.55 |
| Synthesis Example 2-70 | a-4 | — | — | M-37 | 50 | M-42 | 50 | — | — | 8,200 | 1.59 |

### Preparation of Radiation-Sensitive Resin Composition (1)

The acid generating agent (B), the acid diffusion control agent (C), and the organic solvent (D) used in preparation of the radiation-sensitive resin compositions are shown below. In the following Examples and Comparative Examples, unless otherwise specified particularly, the term “parts by mass” means a value, provided that the mass of the polymer (A) used was 100 parts by mass, and the term “mol %” means a value, provided that the mol number of the acid generating agent (B) used was 100 mol %.

(B) Acid Generating Agent

Compounds (hereinafter, may be also referred to as “acid generating agents (B-1) to (B-10)”) represented by the following formulae (B-1) to (B-10) were used as the acid generating agent (B).

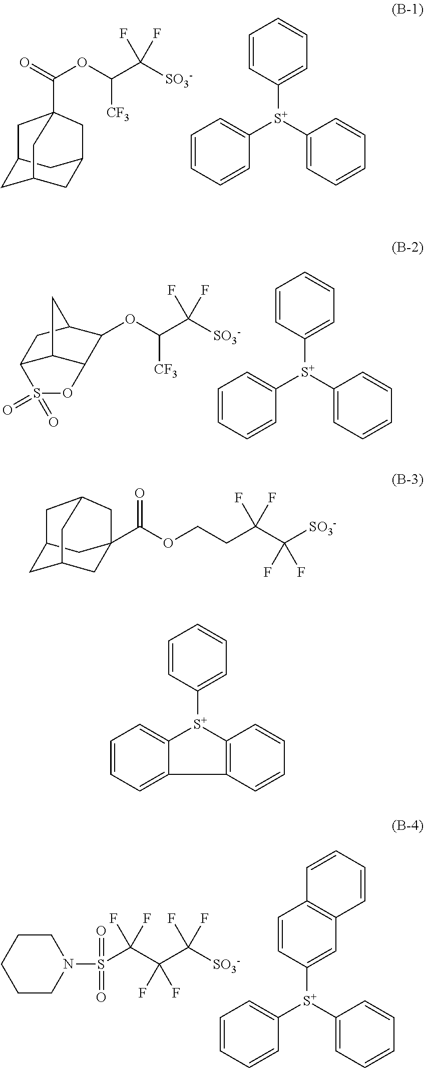

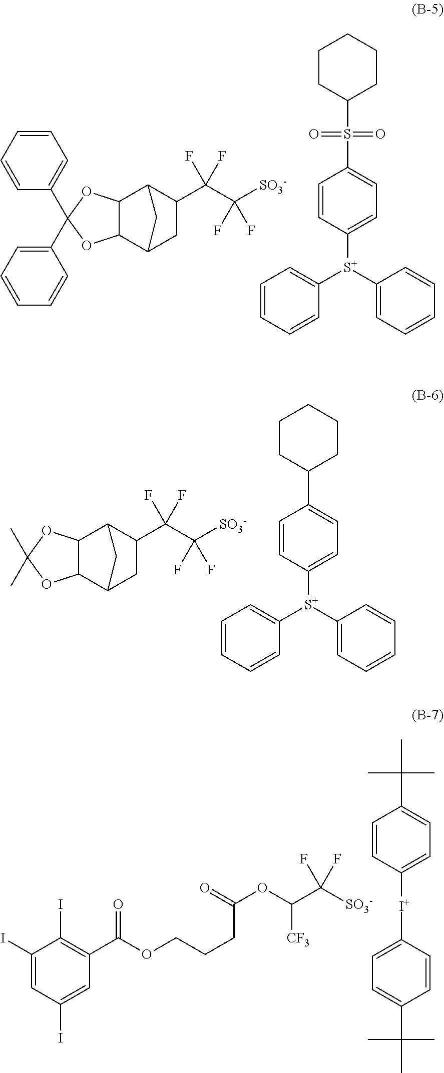

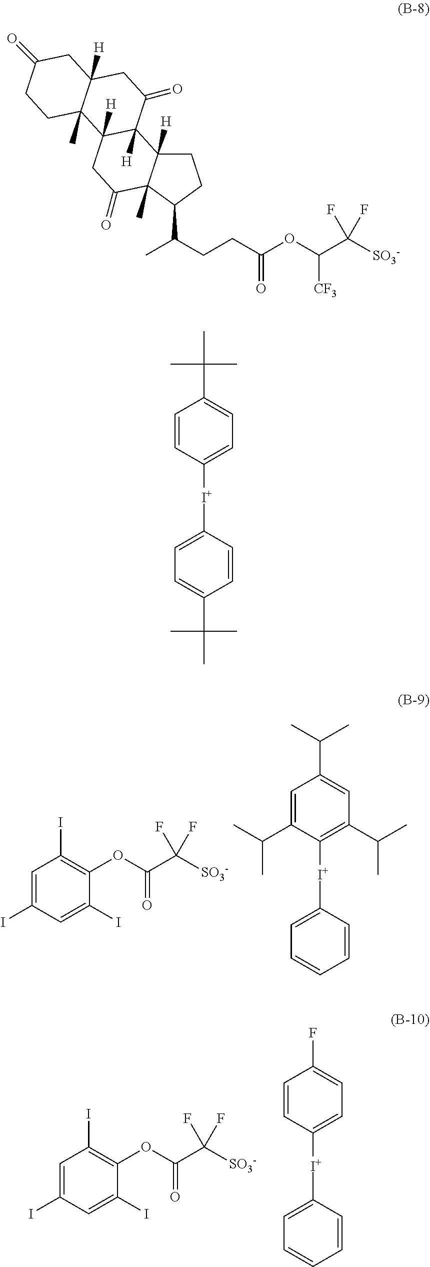

(C) Acid Diffusion Control Agent

Compounds (hereinafter, may be also referred to as “acid diffusion control agents (C-1) to (C-9)”) represented by the following formulae (C-1) to (C-9) were used as the acid diffusion control agent (C).

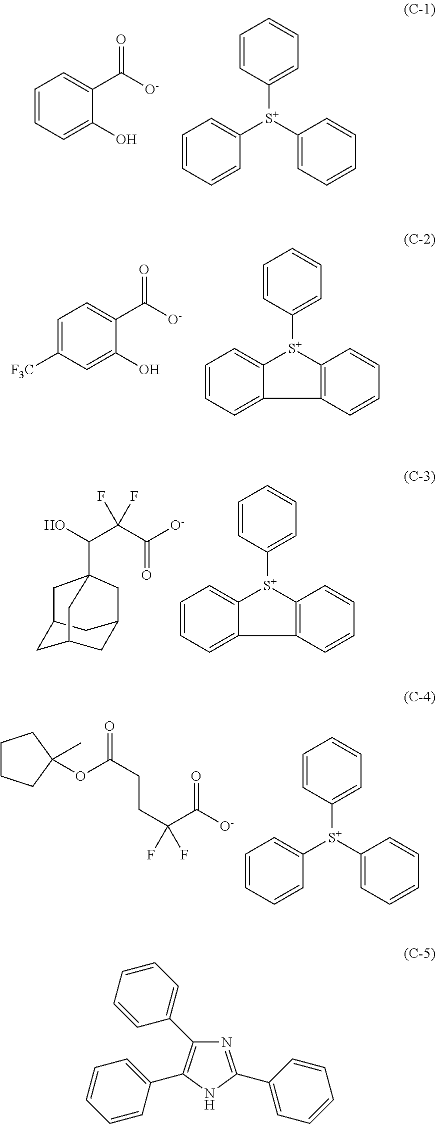

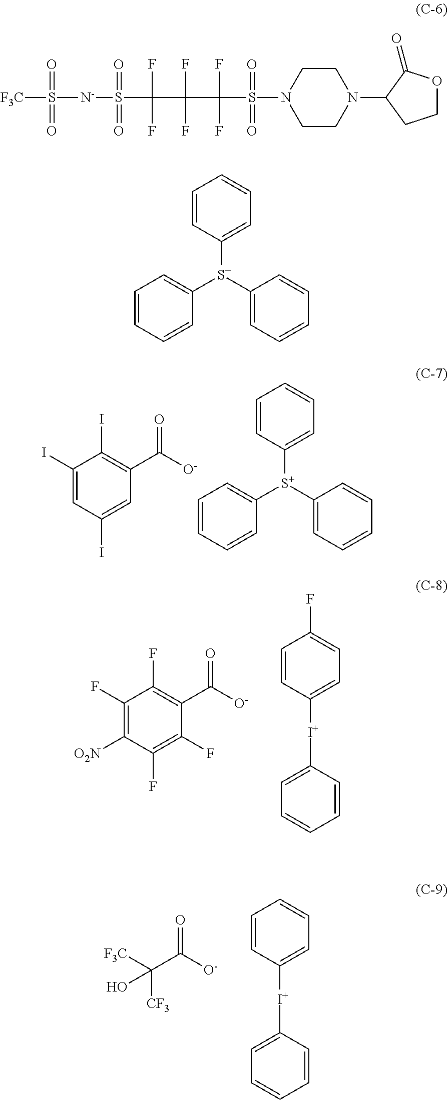

(D) Organic Solvent

The following organic solvents (D-1) and (D-2) were used as the organic solvent (D).
(D-1): propylene glycol monomethyl ether acetate
(D-2): propylene glycol 1-monomethyl ether

### Example 1: Preparation of Radiation-Sensitive Resin Composition (R-1)

A radiation-sensitive resin composition (R-1) was prepared by: mixing 100 parts by mass of (A-1) as the polymer (A), 20 parts by mass of (B-1) as the acid generating agent (B), 20 mol % (C-1) with respect to (B-1) as the acid diffusion control agent (C), and 4,800 parts by mass of (D-1) and 2,000 parts by mass of (D-2) as the organic solvent (D); and filtering a thus resulting mixture through a membrane filter having a pore size of 0.20 m.

### Examples 2 to 85 and Comparative Examples 1 to 4: Preparation of Radiation-Sensitive Resin Compositions (R-2) to (R-85) and (CR-1) to (CR-4)

Radiation-sensitive resin compositions (R-2) to (R-85) and (CR-1) to (CR-4) were prepared in a similar manner to Example 1, except that each component of the type and content shown in Table 2 below was used.

TABLE 2

| | | | | (B) Acid | | (C) Acid diffusion | | | |
|---|---|---|---|---|---|---|---|---|---|
| | Radiation- | (A) Polymer | | generating agent | | control agent | | (D) Organic solvent | |
| | sensitive | | content | | content | | proportion | | content |
| | resin | | (parts by | | (parts by | | (% by | | (parts by |
| | composition | type | mass) | type | mass) | type | mole) | type | mass) |
| Example 1 | R-1 | A-1 | 100 | B-1 | 20 | C-1 | 20 | D-1/D-2 | 4,800/2,000 |
| Example 2 | R-2 | A-2 | 100 | B-1 | 20 | C-1 | 20 | D-1/D-2 | 4,800/2,000 |
| Example 3 | R-3 | A-3 | 100 | B-1 | 20 | C-1 | 20 | D-1/D-2 | 4,800/2,000 |
| Example 4 | R-4 | A-4 | 100 | B-1 | 20 | C-1 | 20 | D-1/D-2 | 4,800/2,000 |
| Example 5 | R-5 | A-5 | 100 | B-1 | 20 | C-1 | 20 | D-1/D-2 | 4,800/2,000 |
| Example 6 | R-6 | A-6 | 100 | B-1 | 20 | C-1 | 20 | D-1/D-2 | 4,800/2,000 |
| Example 7 | R-7 | A-7 | 100 | B-1 | 20 | C-1 | 20 | D-1/D-2 | 4,800/2,000 |
| Example 8 | R-8 | A-8 | 100 | B-1 | 20 | C-1 | 20 | D-1/D-2 | 4,800/2,000 |
| Example 9 | R-9 | A-9 | 100 | B-1 | 20 | C-1 | 20 | D-1/D-2 | 4,800/2,000 |
| Example 10 | R-10 | A-10 | 100 | B-1 | 20 | C-1 | 20 | D-1/D-2 | 4,800/2,000 |
| Example 11 | R-11 | A-11 | 100 | B-1 | 20 | C-1 | 20 | D-1/D-2 | 4,800/2,000 |
| Example 12 | R-12 | A-12 | 100 | B-1 | 20 | C-1 | 20 | D-1/D-2 | 4,800/2,000 |
| Example 13 | R-13 | A-13 | 100 | B-1 | 20 | C-1 | 20 | D-1/D-2 | 4,800/2,000 |
| Example 14 | R-14 | A-14 | 100 | B-1 | 20 | C-1 | 20 | D-1/D-2 | 4,800/2,000 |
| Example 15 | R-15 | A-15 | 100 | B-1 | 20 | C-1 | 20 | D-1/D-2 | 4,800/2,000 |
| Example 16 | R-16 | A-16 | 100 | B-1 | 20 | C-1 | 20 | D-1/D-2 | 4,800/2,000 |
| Example 17 | R-17 | A-17 | 100 | B-1 | 20 | C-1 | 20 | D-1/D-2 | 4,800/2,000 |
| Example 18 | R-18 | A-18 | 100 | B-1 | 20 | C-1 | 20 | D-1/D-2 | 4,800/2,000 |
| Example 19 | R-19 | A-19 | 100 | B-1 | 20 | C-1 | 20 | D-1/D-2 | 4,800/2,000 |
| Example 20 | R-20 | A-20 | 100 | B-1 | 20 | C-1 | 20 | D-1/D-2 | 4,800/2,000 |
| Example 21 | R-21 | A-21 | 100 | B-1 | 20 | C-1 | 20 | D-1/D-2 | 4,800/2,000 |
| Example 22 | R-22 | A-22 | 100 | B-1 | 20 | C-1 | 20 | D-1/D-2 | 4,800/2,000 |
| Example 23 | R-23 | A-23 | 100 | B-1 | 20 | C-1 | 20 | D-1/D-2 | 4,800/2,000 |
| Example 24 | R-24 | A-24 | 100 | B-1 | 20 | C-1 | 20 | D-1/D-2 | 4,800/2,000 |
| Example 25 | R-25 | A-25 | 100 | B-1 | 20 | C-1 | 20 | D-1/D-2 | 4,800/2,000 |
| Example 26 | R-26 | A-26 | 100 | B-1 | 20 | C-1 | 20 | D-1/D-2 | 4,800/2,000 |
| Example 27 | R-27 | A-27 | 100 | B-1 | 20 | C-1 | 20 | D-1/D-2 | 4,800/2,000 |
| Example 28 | R-28 | A-28 | 100 | B-1 | 20 | C-1 | 20 | D-1/D-2 | 4,800/2,000 |
| Example 29 | R-29 | A-29 | 100 | B-1 | 20 | C-1 | 20 | D-1/D-2 | 4,800/2,000 |
| Example 30 | R-30 | A-30 | 100 | B-1 | 20 | C-1 | 20 | D-1/D-2 | 4,800/2,000 |
| Example 31 | R-31 | A-31 | 100 | B-1 | 20 | C-1 | 20 | D-1/D-2 | 4,800/2,000 |
| Example 32 | R-32 | A-32 | 100 | B-1 | 20 | C-1 | 20 | D-1/D-2 | 4,800/2,000 |
| Example 33 | R-33 | A-33 | 100 | B-1 | 20 | C-1 | 20 | D-1/D-2 | 4,800/2,000 |
| Example 34 | R-34 | A-34 | 100 | B-1 | 20 | C-1 | 20 | D-1/D-2 | 4,800/2,000 |
| Example 35 | R-35 | A-35 | 100 | B-1 | 20 | C-1 | 20 | D-1/D-2 | 4,800/2,000 |
| Example 36 | R-36 | A-36 | 100 | B-1 | 20 | C-1 | 20 | D-1/D-2 | 4,800/2,000 |
| Example 37 | R-37 | A-37 | 100 | B-1 | 20 | C-1 | 20 | D-1/D-2 | 4,800/2,000 |
| Example 38 | R-38 | A-38 | 100 | B-1 | 20 | C-1 | 20 | D-1/D-2 | 4,800/2,000 |
| Example 39 | R-39 | A-39 | 100 | B-1 | 20 | C-1 | 20 | D-1/D-2 | 4,800/2,000 |
| Example 40 | R-40 | A-40 | 100 | B-1 | 20 | C-1 | 20 | D-1/D-2 | 4,800/2,000 |
| Example 41 | R-41 | A-41 | 100 | B-1 | 20 | C-1 | 20 | D-1/D-2 | 4,800/2,000 |
| Example 42 | R-42 | A-42 | 100 | B-1 | 20 | C-1 | 20 | D-1/D-2 | 4,800/2,000 |
| Example 43 | R-43 | A-43 | 100 | B-1 | 20 | C-1 | 20 | D-1/D-2 | 4,800/2,000 |
| Example 44 | R-44 | A-44 | 100 | B-1 | 20 | C-1 | 20 | D-1/D-2 | 4,800/2,000 |
| Example 45 | R-45 | A-45 | 100 | B-1 | 20 | C-1 | 20 | D-1/D-2 | 4,800/2,000 |
| Example 46 | R-46 | A-46 | 100 | B-1 | 20 | C-1 | 20 | D-1/D-2 | 4,800/2,000 |
| Example 47 | R-47 | A-47 | 100 | B-1 | 20 | C-1 | 20 | D-1/D-2 | 4,800/2,000 |
| Example 48 | R-48 | A-48 | 100 | B-1 | 20 | C-1 | 20 | D-1/D-2 | 4,800/2,000 |
| Example 49 | R-49 | A-49 | 100 | B-1 | 20 | C-1 | 20 | D-1/D-2 | 4,800/2,000 |
| Example 50 | R-50 | A-50 | 100 | B-1 | 20 | C-1 | 20 | D-1/D-2 | 4,800/2,000 |
| Example 51 | R-51 | A-51 | 100 | B-1 | 20 | C-1 | 20 | D-1/D-2 | 4,800/2,000 |
| Example 52 | R-52 | A-52 | 100 | B-1 | 20 | C-1 | 20 | D-1/D-2 | 4,800/2,000 |
| Example 53 | R-53 | A-53 | 100 | B-1 | 20 | C-1 | 20 | D-1/D-2 | 4,800/2,000 |
| Example 54 | R-54 | A-54 | 100 | B-1 | 20 | C-1 | 20 | D-1/D-2 | 4,800/2,000 |
| Example 55 | R-55 | A-55 | 100 | B-1 | 20 | C-1 | 20 | D-1/D-2 | 4,800/2,000 |
| Example 56 | R-56 | A-56 | 100 | B-1 | 20 | C-1 | 20 | D-1/D-2 | 4,800/2,000 |
| Example 57 | R-57 | A-57 | 100 | B-1 | 20 | C-1 | 20 | D-1/D-2 | 4,800/2,000 |
| Example 58 | R-58 | A-58 | 100 | B-1 | 20 | C-1 | 20 | D-1/D-2 | 4,800/2,000 |
| Example 59 | R-59 | A-59 | 100 | B-1 | 20 | C-1 | 20 | D-1/D-2 | 4,800/2,000 |
| Example 60 | R-60 | A-60 | 100 | B-1 | 20 | C-1 | 20 | D-1/D-2 | 4,800/2,000 |
| Example 61 | R-61 | A-61 | 100 | B-1 | 20 | C-1 | 20 | D-1/D-2 | 4,800/2,000 |
| Example 62 | R-62 | A-62 | 100 | B-1 | 20 | C-1 | 20 | D-1/D-2 | 4,800/2,000 |
| Example 63 | R-63 | A-63 | 100 | B-1 | 20 | C-1 | 20 | D-1/D-2 | 4,800/2,000 |
| Example 64 | R-64 | A-64 | 100 | B-1 | 20 | C-1 | 20 | D-1/D-2 | 4,800/2,000 |
| Example 65 | R-65 | A-65 | 100 | B-1 | 20 | C-1 | 20 | D-1/D-2 | 4,800/2,000 |
| Example 66 | R-66 | A-1 | 100 | B-1 | 30 | C-1 | 20 | D-1/D-2 | 4,800/2,000 |
| Example 67 | R-67 | A-1 | 100 | B-1 | 40 | C-1 | 20 | D-1/D-2 | 4,800/2,000 |
| Example 68 | R-68 | A-1 | 100 | B-2 | 20 | C-1 | 20 | D-1/D-2 | 4,800/2,000 |
| Example 69 | R-69 | A-1 | 100 | B-3 | 20 | C-1 | 20 | D-1/D-2 | 4,800/2,000 |
| Example 70 | R-70 | A-1 | 100 | B-4 | 20 | C-1 | 20 | D-1/D-2 | 4,800/2,000 |
| Example 71 | R-71 | A-1 | 100 | B-5 | 20 | C-1 | 20 | D-1/D-2 | 4,800/2,000 |
| Example 72 | R-72 | A-1 | 100 | B-6 | 20 | C-1 | 20 | D-1/D-2 | 4,800/2,000 |
| Example 73 | R-73 | A-1 | 100 | B-1 | 20 | C-2 | 20 | D-1/D-2 | 4,800/2,000 |
| Example 74 | R-74 | A-1 | 100 | B-1 | 20 | C-3 | 20 | D-1/D-2 | 4,800/2,000 |
| Example 75 | R-75 | A-1 | 100 | B-1 | 20 | C-4 | 20 | D-1/D-2 | 4,800/2,000 |
| Example 76 | R-76 | A-66 | 100 | B-1 | 20 | C-1 | 20 | D-1/D-2 | 4,800/2,000 |
| Example 77 | R-77 | A-1 | 100 | B-1 | 20 | C-5 | 20 | D-1/D-2 | 4,800/2,000 |
| Example 78 | R-78 | A-1 | 100 | B-1 | 20 | C-6 | 20 | D-1/D-2 | 4,800/2,000 |
| Example 79 | R-79 | A-1 | 100 | B-7 | 20 | C-1 | 20 | D-1/D-2 | 4,800/2,000 |
| Example 80 | R-80 | A-1 | 100 | B-8 | 20 | C-2 | 20 | D-1/D-2 | 4,800/2,000 |
| Example 81 | R-81 | A-1 | 100 | B-9 | 20 | C-3 | 20 | D-1/D-2 | 4,800/2,000 |
| Example 82 | R-82 | A-1 | 100 | B-10 | 20 | C-3 | 20 | D-1/D-2 | 4,800/2,000 |
| Example 83 | R-83 | A-1 | 100 | B-1 | 20 | C-7 | 20 | D-1/D-2 | 4,800/2,000 |
| Example 84 | R-84 | A-1 | 100 | B-1 | 20 | C-8 | 20 | D-1/D-2 | 4,800/2,000 |
| Example 85 | R-85 | A-1 | 100 | B-1 | 20 | C-9 | 20 | D-1/D-2 | 4,800/2,000 |
| Comparative | CR-1 | a-1 | 100 | B-1 | 20 | C-1 | 20 | D-1/D-2 | 4,800/2,000 |
| Example 1 | | | | | | | | | |
| Comparative | CR-2 | a-2 | 100 | B-1 | 20 | C-1 | 20 | D-1/D-2 | 4,800/2,000 |
| Example 2 | | | | | | | | | |
| Comparative | CR-3 | a-3 | 100 | B-1 | 20 | C-1 | 20 | D-1/D-2 | 4,800/2,000 |
| Example 3 | | | | | | | | | |
| Comparative | CR-4 | a-4 | 100 | B-1 | 20 | C-1 | 20 | D-1/D-2 | 4,800/2,000 |
| Example 4 | | | | | | | | | |

### Resist Pattern Formation (EUV Exposure, Alkali Development)

On a 12-inch silicon wafer surface provided thereon with an underlayer film (“AL412,” available from Brewer Science, Inc.) having an average thickness of 20 nm, the radiation-sensitive resin compositions prepared as described above were each applied using a spin coater (“CLEAN TRACK ACT 12,” available from Tokyo Electron Limited). Next, prebaking (PB) was conducted at 130° C. for 60 sec, followed by cooling at 23° C. for 30 sec to form a resist film having an average thickness of 50 nm. Next, the resist film was irradiated with EUV light using an EUV scanner (“NXE3300”, available from ASML Co.) with NA of 0.33 under an illumination condition of Conventional s=0.89 and with a mask of imecDEFECT32FFR02. After the irradiating, the resist film was subjected to post exposure baking (PEB) at 130° C. for 60 sec. Subsequently, a positive-tone resist pattern with 32 nm lines-and-spaces was formed by development at 23° C. for 30 sec by using a 2.38% by mass aqueous TMAH solution as an alkaline developer solution.

### Evaluations

Each of the resist patterns formed in the section “Resist Pattern Formation (EUV Exposure, Alkali Development)” was evaluated on sensitivity, LWR performance, and process window in accordance with the following methods. A scanning electron microscope (“CG-4100,” available from Hitachi High-Technologies Corporation) was used for line-width measurement of the resist pattern. The results of the evaluations are shown in Table 3 below.

Sensitivity

An exposure dose at which a 32 nm line-and-space pattern was formed in the above section “Resist Pattern Formation (EUV Exposure, Alkali Development)” was defined as an optimum exposure dose, and this optimum exposure dose was adopted as Eop (unit: mJ/cm²). The sensitivity being more favorable is indicated by the Eop value being smaller.

LWR Performance

The resist patterns were observed from above using the scanning electron microscope. Line widths were measured at 50 points in total at arbitrary locations, and then a 3 Sigma value was determined from distribution of the measurements and was defined as LWR (unit: nm). The value of the LWR being smaller reveals less unevenness of the lines, indicating better LWR performance.

Process Window

The “process window” as referred to herein means the range of resist dimensions at which a pattern having no bridge defects or collapses can be formed. Using a mask for forming 32 nm lines-and-spaces (1 L/1 S), patterns were formed with low-exposure doses to high-exposure doses. In general, defects in bridge formation and the like can be found in patterns in the case of the low-exposure dose, and defects such as pattern collapses can be found in the case of the high-exposure dose. The difference between the maximum value and the minimum value of resist dimensions at which no such defects were found was considered to be the CD (Critical Dimension) margin (unit: nm). The CD margin being large reveals a broader process window, and is favorable.

TABLE 3

| | Radiation- | Eop | | CD |
|---|---|---|---|---|
| | sensitive resin | (mJ/ | LWR | margin |
| | composition | cm²) | (nm) | (nm) |
| Example 1 | R-1 | 27 | 3.5 | 37 |
| Example 2 | R-2 | 26 | 3.7 | 36 |
| Example 3 | R-3 | 24 | 3.8 | 33 |
| Example 4 | R-4 | 25 | 3.6 | 39 |
| Example 5 | R-5 | 24 | 3.5 | 36 |
| Example 6 | R-6 | 24 | 3.7 | 37 |
| Example 7 | R-7 | 22 | 3.9 | 38 |
| Example 8 | R-8 | 29 | 3.3 | 31 |
| Example 9 | R-9 | 23 | 3.5 | 35 |
| Example 10 | R-10 | 27 | 3.4 | 32 |
| Example 11 | R-11 | 30 | 3.8 | 31 |
| Example 12 | R-12 | 26 | 3.6 | 33 |
| Example 13 | R-13 | 29 | 3.7 | 30 |
| Example 14 | R-14 | 25 | 3.9 | 31 |
| Example 15 | R-15 | 26 | 3.7 | 33 |
| Example 16 | R-16 | 25 | 3.7 | 35 |
| Example 17 | R-17 | 27 | 3.5 | 31 |
| Example 18 | R-18 | 26 | 3.7 | 34 |
| Example 19 | R-19 | 24 | 3.9 | 30 |
| Example 20 | R-20 | 30 | 3.8 | 33 |
| Example 21 | R-21 | 26 | 3.7 | 35 |
| Example 22 | R-22 | 27 | 3.6 | 32 |
| Example 23 | R-23 | 30 | 3.9 | 34 |
| Example 24 | R-24 | 26 | 3.5 | 30 |
| Example 25 | R-25 | 29 | 3.8 | 35 |
| Example 26 | R-26 | 28 | 3.7 | 31 |
| Example 27 | R-27 | 25 | 3.7 | 36 |
| Example 28 | R-28 | 30 | 3.9 | 36 |
| Example 29 | R-29 | 29 | 3.8 | 34 |
| Example 30 | R-30 | 24 | 3.9 | 30 |
| Example 31 | R-31 | 26 | 3.8 | 33 |
| Example 32 | R-32 | 30 | 3.7 | 31 |
| Example 33 | R-33 | 30 | 3.3 | 31 |
| Example 34 | R-34 | 29 | 3.4 | 35 |
| Example 35 | R-35 | 28 | 3.5 | 37 |
| Example 36 | R-36 | 25 | 3.7 | 35 |
| Example 37 | R-37 | 24 | 3.9 | 32 |
| Example 38 | R-38 | 26 | 3.8 | 32 |
| Example 39 | R-39 | 29 | 3.5 | 35 |
| Example 40 | R-40 | 29 | 3.3 | 37 |
| Example 41 | R-41 | 27 | 3.7 | 32 |
| Example 42 | R-42 | 30 | 3.3 | 38 |
| Example 43 | R-43 | 27 | 3.8 | 36 |
| Example 44 | R-44 | 26 | 3.6 | 35 |
| Example 45 | R-45 | 25 | 3.7 | 35 |
| Example 46 | R-46 | 23 | 3.9 | 31 |
| Example 47 | R-47 | 30 | 3.8 | 33 |
| Example 48 | R-48 | 27 | 3.6 | 34 |
| Example 49 | R-49 | 24 | 3.8 | 30 |
| Example 50 | R-50 | 30 | 3.4 | 33 |
| Example 51 | R-51 | 26 | 3.5 | 36 |
| Example 52 | R-52 | 27 | 3.8 | 31 |
| Example 53 | R-53 | 29 | 3.8 | 32 |
| Example 54 | R-54 | 29 | 3.8 | 33 |
| Example 55 | R-55 | 30 | 4.0 | 31 |
| Example 56 | R-56 | 28 | 3.9 | 33 |
| Example 57 | R-57 | 30 | 3.9 | 34 |
| Example 58 | R-58 | 27 | 3.8 | 32 |
| Example 59 | R-59 | 29 | 3.6 | 35 |
| Example 60 | R-60 | 30 | 3.8 | 32 |
| Example 61 | R-61 | 35 | 3.7 | 23 |
| Example 62 | R-62 | 33 | 3.9 | 28 |
| Example 63 | R-63 | 29 | 3.6 | 35 |
| Example 64 | R-64 | 29 | 3.5 | 35 |
| Example 65 | R-65 | 27 | 3.4 | 36 |
| Example 66 | R-66 | 24 | 3.7 | 35 |
| Example 67 | R-67 | 22 | 3.8 | 34 |
| Example 68 | R-68 | 28 | 3.7 | 36 |
| Example 69 | R-69 | 30 | 3.8 | 32 |
| Example 70 | R-70 | 25 | 3.9 | 33 |
| Example 71 | R-71 | 23 | 3.7 | 31 |
| Example 72 | R-72 | 29 | 3.5 | 34 |
| Example 73 | R-73 | 27 | 3.7 | 32 |
| Example 74 | R-74 | 29 | 3.4 | 33 |
| Example 75 | R-75 | 28 | 3.6 | 35 |
| Example 76 | R-76 | 25 | 3.6 | 28 |
| Example 77 | R-77 | 38 | 4.4 | 25 |
| Example 78 | R-78 | 28 | 4.2 | 26 |
| Example 79 | R-79 | 26 | 4.4 | 27 |
| Example 80 | R-80 | 25 | 4.3 | 29 |
| Example 81 | R-81 | 27 | 4.7 | 25 |
| Example 82 | R-82 | 26 | 4.4 | 26 |
| Example 83 | R-83 | 25 | 4.6 | 28 |
| Example 84 | R-84 | 28 | 4.8 | 27 |
| Example 85 | R-85 | 29 | 4.4 | 29 |
| Comparative | CR-1 | 31 | 4.3 | 33 |
| Example 1 | | | | |
| Comparative | CR-2 | 29 | 4.1 | 31 |
| Example 2 | | | | |
| Comparative | CR-3 | 28 | 3.8 | 26 |
| Example 3 | | | | |
| Comparative | CR-4 | 26 | 4.3 | 22 |
| Example 4 | | | | |

### Example 86: Preparation of Radiation-Sensitive Resin Composition (R-86)

A radiation-sensitive resin composition (R-86) was prepared by: mixing 100 parts by mass of (A-1) as the polymer (A), 10 parts by mass of (B-1) as the acid generating agent (B), 40 mol % (C-1) with respect to (B-1) as the acid diffusion control agent (C), and 4,800 parts by mass of (D-1) and 2,000 parts by mass of (D-2) as the organic solvent (D); and filtering a resulting mixture through a membrane filter having a pore size of 0.20 km.

### Examples 87 to 139 and Comparative Examples 5 to 8: Preparation of Radiation-Sensitive Resin Compositions (R-87) to (R-139) and (CR-5) to (CR-8)

Radiation-sensitive resin compositions (R-87) to (R-139) and (CR-5) to (CR-8) were prepared in a similar manner to Example 86, except that each component of the type and content shown in Table 4 below was used.

TABLE 4

| | | | | (B) Acid | | (C) Acid diffusion | | | |
|---|---|---|---|---|---|---|---|---|---|
| | Radiation- | (A) Polymer | | generating agent | | control agent | | (D) Organic solvent | |
| | sensitive | | content | | content | | proportion | | content |
| | resin | | (parts by | | (parts by | | (% by | | (parts by |
| | composition | type | mass) | type | mass) | type | mole) | type | mass) |
| Example 86 | R-86 | A-1 | 100 | B-1 | 10 | C-1 | 40 | D-1/D-2 | 4,800/2,000 |
| Example 87 | R-87 | A-2 | 100 | B-1 | 10 | C-1 | 40 | D-1/D-2 | 4,800/2,000 |
| Example 88 | R-88 | A-3 | 100 | B-1 | 10 | C-1 | 40 | D-1/D-2 | 4,800/2,000 |
| Example 89 | R-89 | A-4 | 100 | B-1 | 10 | C-1 | 40 | D-1/D-2 | 4,800/2,000 |
| Example 90 | R-90 | A-5 | 100 | B-1 | 10 | C-1 | 40 | D-1/D-2 | 4,800/2,000 |
| Example 91 | R-91 | A-6 | 100 | B-1 | 10 | C-1 | 40 | D-1/D-2 | 4,800/2,000 |
| Example 92 | R-92 | A-7 | 100 | B-1 | 10 | C-1 | 40 | D-1/D-2 | 4,800/2,000 |
| Example 93 | R-93 | A-8 | 100 | B-1 | 10 | C-1 | 40 | D-1/D-2 | 4,800/2,000 |
| Example 94 | R-94 | A-9 | 100 | B-1 | 10 | C-1 | 40 | D-1/D-2 | 4,800/2,000 |
| Example 95 | R-95 | A-10 | 100 | B-1 | 10 | C-1 | 40 | D-1/D-2 | 4,800/2,000 |
| Example 96 | R-96 | A-11 | 100 | B-1 | 10 | C-1 | 40 | D-1/D-2 | 4,800/2,000 |
| Example 97 | R-97 | A-12 | 100 | B-1 | 10 | C-1 | 40 | D-1/D-2 | 4,800/2,000 |
| Example 98 | R-98 | A-13 | 100 | B-1 | 10 | C-1 | 40 | D-1/D-2 | 4,800/2,000 |
| Example 99 | R-99 | A-14 | 100 | B-1 | 10 | C-1 | 40 | D-1/D-2 | 4,800/2,000 |
| Example 100 | R-100 | A-15 | 100 | B-1 | 10 | C-1 | 40 | D-1/D-2 | 4,800/2,000 |
| Example 101 | R-101 | A-16 | 100 | B-1 | 10 | C-1 | 40 | D-1/D-2 | 4,800/2,000 |
| Example 102 | R-102 | A-17 | 100 | B-1 | 10 | C-1 | 40 | D-1/D-2 | 4,800/2,000 |
| Example 103 | R-103 | A-18 | 100 | B-1 | 10 | C-1 | 40 | D-1/D-2 | 4,800/2,000 |
| Example 104 | R-104 | A-19 | 100 | B-1 | 10 | C-1 | 40 | D-1/D-2 | 4,800/2,000 |
| Example 105 | R-105 | A-20 | 100 | B-1 | 10 | C-1 | 40 | D-1/D-2 | 4,800/2,000 |
| Example 106 | R-106 | A-21 | 100 | B-1 | 10 | C-1 | 40 | D-1/D-2 | 4,800/2,000 |
| Example 107 | R-107 | A-22 | 100 | B-1 | 10 | C-1 | 40 | D-1/D-2 | 4,800/2,000 |
| Example 108 | R-108 | A-23 | 100 | B-1 | 10 | C-1 | 40 | D-1/D-2 | 4,800/2,000 |
| Example 109 | R-109 | A-24 | 100 | B-1 | 10 | C-1 | 40 | D-1/D-2 | 4,800/2,000 |
| Example 110 | R-110 | A-25 | 100 | B-1 | 10 | C-1 | 40 | D-1/D-2 | 4,800/2,000 |
| Example 111 | R-111 | A-26 | 100 | B-1 | 10 | C-1 | 40 | D-1/D-2 | 4,800/2,000 |
| Example 112 | R-112 | A-27 | 100 | B-1 | 10 | C-1 | 40 | D-1/D-2 | 4,800/2,000 |
| Example 113 | R-113 | A-28 | 100 | B-1 | 10 | C-1 | 40 | D-1/D-2 | 4,800/2,000 |
| Example 114 | R-114 | A-29 | 100 | B-1 | 10 | C-1 | 40 | D-1/D-2 | 4,800/2,000 |
| Example 115 | R-115 | A-30 | 100 | B-1 | 10 | C-1 | 40 | D-1/D-2 | 4,800/2,000 |
| Example 116 | R-116 | A-31 | 100 | B-1 | 10 | C-1 | 40 | D-1/D-2 | 4,800/2,000 |
| Example 117 | R-117 | A-32 | 100 | B-1 | 10 | C-1 | 40 | D-1/D-2 | 4,800/2,000 |
| Example 118 | R-118 | A-61 | 100 | B-1 | 10 | C-1 | 40 | D-1/D-2 | 4,800/2,000 |
| Example 119 | R-119 | A-62 | 100 | B-1 | 10 | C-1 | 40 | D-1/D-2 | 4,800/2,000 |
| Example 120 | R-120 | A-1 | 100 | B-1 | 20 | C-1 | 40 | D-1/D-2 | 4,800/2,000 |
| Example 121 | R-121 | A-1 | 100 | B-1 | 30 | C-1 | 40 | D-1/D-2 | 4,800/2,000 |
| Example 122 | R-122 | A-1 | 100 | B-2 | 10 | C-1 | 40 | D-1/D-2 | 4,800/2,000 |
| Example 123 | R-123 | A-1 | 100 | B-3 | 10 | C-1 | 40 | D-1/D-2 | 4,800/2,000 |
| Example 124 | R-124 | A-1 | 100 | B-4 | 10 | C-1 | 40 | D-1/D-2 | 4,800/2,000 |
| Example 125 | R-125 | A-1 | 100 | B-5 | 10 | C-1 | 40 | D-1/D-2 | 4,800/2,000 |
| Example 126 | R-126 | A-1 | 100 | B-6 | 10 | C-1 | 40 | D-1/D-2 | 4,800/2,000 |
| Example 127 | R-127 | A-1 | 100 | B-1 | 10 | C-2 | 40 | D-1/D-2 | 4,800/2,000 |
| Example 128 | R-128 | A-1 | 100 | B-1 | 10 | C-3 | 40 | D-1/D-2 | 4,800/2,000 |
| Example 129 | R-129 | A-1 | 100 | B-2 | 10 | C-4 | 40 | D-1/D-2 | 4,800/2,000 |
| Example 130 | R-130 | A-66 | 100 | B-3 | 10 | C-5 | 40 | D-1/D-2 | 4,800/2,000 |
| Example 131 | R-131 | A-1 | 100 | B-4 | 10 | C-6 | 40 | D-1/D-2 | 4,800/2,000 |
| Example 132 | R-132 | A-1 | 100 | B-5 | 10 | C-7 | 40 | D-1/D-2 | 4,800/2,000 |
| Example 133 | R-133 | A-1 | 100 | B-7 | 10 | C-1 | 40 | D-1/D-2 | 4,800/2,000 |
| Example 134 | R-134 | A-1 | 100 | B-8 | 10 | C-1 | 40 | D-1/D-2 | 4,800/2,000 |
| Example 135 | R-135 | A-1 | 100 | B-9 | 10 | C-1 | 40 | D-1/D-2 | 4,800/2,000 |
| Example 136 | R-136 | A-1 | 100 | B-10 | 10 | C-1 | 40 | D-1/D-2 | 4,800/2,000 |
| Example 137 | R-137 | A-1 | 100 | B-1 | 10 | C-7 | 40 | D-1/D-2 | 4,800/2,000 |
| Example 138 | R-138 | A-1 | 100 | B-1 | 10 | C-8 | 40 | D-1/D-2 | 4,800/2,000 |
| Example 139 | R-139 | A-1 | 100 | B-1 | 10 | C-9 | 40 | D-1/D-2 | 4,800/2,000 |
| Comparative | CR-5 | a-1 | 100 | B-1 | 10 | C-1 | 40 | D-1/D-2 | 4,800/2,000 |
| Example 5 | | | | | | | | | |
| Comparative | CR-6 | a-2 | 100 | B-1 | 10 | C-1 | 40 | D-1/D-2 | 4,800/2,000 |
| Example 6 | | | | | | | | | |
| Comparative | CR-7 | a-3 | 100 | B-1 | 10 | C-1 | 40 | D-1/D-2 | 4,800/2,000 |
| Example 7 | | | | | | | | | |
| Comparative | CR-8 | a-4 | 100 | B-1 | 10 | C-1 | 40 | D-1/D-2 | 4,800/2,000 |
| Example 8 | | | | | | | | | |

### Resist Pattern Formation (EUV Exposure, Organic Solvent Development)

The radiation-sensitive resin compositions prepared as described above were applied by using the aforementioned spin-coater, on a 12-inch silicon wafer which had been subjected to an HMDS treatment. Next, PB was conducted at 130° C. for 60 sec, followed by cooling at 23° C. for 30 sec to form a resist film having a film thickness of 30 nm. Next, the resist film was irradiated with EUV light through a 20H40P contact hole mask pattern under optical conditions involving Annular (σ=0.89/0.70). After the irradiating, PEB was carried out at 130° C. for 60 sec. Thereafter, the resist film was subjected to development with an organic solvent at 23° C. for 10 sec using n-butyl acetate as an organic solvent developer solution, and dried to form a negative-tone resist pattern (hereinafter, may be also referred to as “20H40P contact hole pattern”) having hole diameters of 20 nm and pitch of 40 nm.

### Evaluations

Each of the resist patterns formed in the section “Resist Pattern Formation (EUV Exposure, Organic Solvent Development)” was evaluated on sensitivity and CDU performance in accordance with the following methods. The results of the evaluations are shown in Table 5 below.

Sensitivity

An exposure dose at which the 20H40P contact hole pattern was formed in the above section “Resist Pattern Formation (EUV Exposure, Organic Solvent Development)” was defined as an optimum exposure dose, and this optimum exposure dose was adopted as Eop (unit: mJ/cm²). The Eop value being smaller indicates more favorable sensitivity.

CDU Performance

The resist patterns were observed from above using the scanning electron microscope, and hole diameters were measured at 27,000 points in total at arbitrary locations to determine a 3 Sigma value from distribution of the measurement values and defined as “CDU” (unit: nm). The CDU value being smaller indicates more favorable CDU performance, revealing less variance of the hole diameters in greater ranges.

TABLE 5

| | Radiation- | Eop | |
|---|---|---|---|
| | sensitive resin | (mJ/ | CDU |
| | composition | cm²) | (nm) |
| Example 86 | R-86 | 85 | 3.2 |
| Example 87 | R-87 | 83 | 3.3 |
| Example 88 | R-88 | 80 | 3.5 |
| Example 89 | R-89 | 81 | 3.1 |
| Example 90 | R-90 | 80 | 3.2 |
| Example 91 | R-91 | 81 | 3.4 |
| Example 92 | R-92 | 83 | 3.5 |
| Example 93 | R-93 | 82 | 3.4 |
| Example 94 | R-94 | 80 | 3.4 |
| Example 95 | R-95 | 86 | 3.5 |
| Example 96 | R-96 | 88 | 3.4 |
| Example 97 | R-97 | 86 | 3.4 |
| Example 98 | R-98 | 88 | 3.2 |
| Example 99 | R-99 | 87 | 3.1 |
| Example 100 | R-100 | 83 | 3.2 |
| Example 101 | R-101 | 85 | 3.4 |
| Example 102 | R-102 | 86 | 3.5 |
| Example 103 | R-103 | 84 | 3.5 |
| Example 104 | R-104 | 85 | 3.5 |
| Example 105 | R-105 | 86 | 3.4 |
| Example 106 | R-106 | 88 | 3.3 |
| Example 107 | R-107 | 87 | 3.4 |
| Example 108 | R-108 | 89 | 3.5 |
| Example 109 | R-109 | 83 | 3.5 |
| Example 110 | R-110 | 86 | 3.2 |
| Example 111 | R-111 | 87 | 3.4 |
| Example 112 | R-112 | 85 | 3.3 |
| Example 113 | R-113 | 82 | 3.3 |
| Example 114 | R-114 | 84 | 3.4 |
| Example 115 | R-115 | 82 | 3.5 |
| Example 116 | R-116 | 87 | 3.2 |
| Example 117 | R-117 | 89 | 3.1 |
| Example 118 | R-118 | 96 | 3.8 |
| Example 119 | R-119 | 97 | 4.1 |
| Example 120 | R-120 | 84 | 3.5 |
| Example 121 | R-121 | 82 | 3.5 |
| Example 122 | R-122 | 87 | 3.3 |
| Example 123 | R-123 | 90 | 3.0 |
| Example 124 | R-124 | 85 | 3.4 |
| Example 125 | R-125 | 83 | 3.4 |
| Example 126 | R-126 | 89 | 3.2 |
| Example 127 | R-127 | 86 | 3.3 |
| Example 128 | R-128 | 88 | 3.2 |
| Example 129 | R-129 | 87 | 3.4 |
| Example 130 | R-130 | 88 | 3.7 |
| Example 131 | R-131 | 98 | 4.2 |
| Example 132 | R-132 | 87 | 4.5 |
| Example 133 | R-133 | 85 | 4.8 |
| Example 134 | R-134 | 85 | 4.1 |
| Example 135 | R-135 | 89 | 4.0 |
| Example 136 | R-136 | 88 | 4.1 |
| Example 137 | R-137 | 86 | 4.4 |
| Example 138 | R-138 | 91 | 4.9 |
| Example 139 | R-139 | 90 | 4.5 |
| Comparative | CR-5 | 93 | 4.6 |
| Example 5 | | | |
| Comparative | CR-6 | 89 | 4.8 |
| Example 6 | | | |
| Comparative | CR-7 | 88 | 3.9 |
| Example 7 | | | |
| Comparative | CR-8 | 86 | 4.3 |
| Example 8 | | | |

The radiation-sensitive resin composition and the method of forming a resist pattern of the embodiments of the present invention enable formation of a resist pattern with favorable sensitivity to exposure light, superiority in LWR performance and CDU performance, and a broad process window. The polymer of the still another embodiment of the present invention can be suitably used as a component of the radiation-sensitive resin composition of the one embodiment of the present invention. The compound of the yet another embodiment of the present invention can be suitably used as a monomer for synthesizing the polymer of the still another embodiment of the present invention. Therefore, these can be suitably used in manufacturing processes of semiconductor devices and the like, in which further progress of miniaturization is expected in the future.

Obviously, numerous modifications and variations of the present invention(s) are possible in light of the above teachings. It is therefore to be understood that within the scope of the appended claims, the invention(s) may be practiced otherwise than as specifically described herein.

1: A radiation-sensitive resin composition comprising:
a polymer comprising a first structural unit represented by formula (1); and
a radiation-sensitive acid generator, 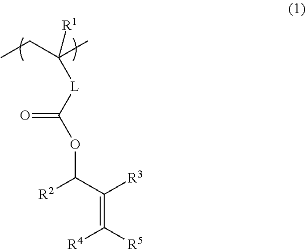
wherein, in the formula (1),
R¹ represents a hydrogen atom, a fluorine atom, a methyl group, or a trifluoromethyl group;
R², R³, and R⁴ each independently represent a hydrogen atom or a monovalent organic group having 1 to 20 carbon atoms;
R⁵ represents a monovalent organic group having 1 to 20 carbon atoms; and
L represents a single bond or a divalent organic group having 1 to 20 carbon atoms.

2: The radiation-sensitive resin composition according to claim 1, wherein R² represents the monovalent organic group having 1 to 20 carbon atoms.

3: The radiation-sensitive resin composition according to claim 1, wherein the organic group which is represented by R² is a monovalent hydrocarbon group having 1 to 20 carbon atoms, or a group obtained by substituting a part or all of hydrogen atoms contained in the monovalent hydrocarbon group with a monovalent heteroatom-containing group.

4. The radiation-sensitive resin composition according to claim 1, wherein R³ represents a hydrogen atom or a monovalent hydrocarbon group having 1 to 20 carbon atoms.

5: The radiation-sensitive resin composition according to claim 1, wherein R⁴ represents a hydrogen atom or a monovalent hydrocarbon group having 1 to 20 carbon atoms.

6: The radiation-sensitive resin composition according to claim 1, wherein R⁵ represents a monovalent hydrocarbon group having 1 to 20 carbon atoms.

7: The radiation-sensitive resin composition according to claim 1, wherein the polymer further comprises a second structural unit comprising a phenolic hydroxy group.

8: The radiation-sensitive resin composition according to claim 1, wherein the polymer further comprises a third structural unit which is a structural unit other than the first structural unit and comprises an acid-labile group.

9: A method of forming a resist pattern, the method comprising:
forming a resist film directly or indirectly on a substrate by applying the radiation-sensitive resin composition according to claim 1;
exposing the resist film; and
developing the resist film exposed.

10: A polymer comprising a first structural unit represented by formula (1): 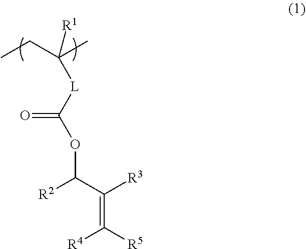
wherein, in the formula (1),
R¹ represents a hydrogen atom, a fluorine atom, a methyl group, or a trifluoromethyl group;
R², R³, and R⁴ each independently represent a hydrogen atom or a monovalent organic group having 1 to 20 carbon atoms;
R⁵ represents a monovalent organic group having 1 to 20 carbon atoms; and
L represents a single bond or a divalent organic group having 1 to 20 carbon atoms.

11: A compound represented by formula (1′): 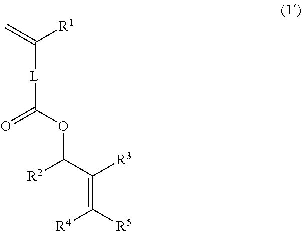
wherein, in the formula (1′),
R¹ represents a hydrogen atom, a fluorine atom, a methyl group, or a trifluoromethyl group;
R², R³, and R⁴ each independently represent a hydrogen atom or a monovalent organic group having 1 to 20 carbon atoms;
R⁵ represents a monovalent organic group having 1 to 20 carbon atoms; and
L represents a single bond or a divalent organic group having 1 to 20 carbon atoms.
